SEQUENCE LISTING (6 sequences)

SEQ ID NO: 1 - protein, 1023 aa, Homo sapiens
MGKGVGRDKYEPAAVSEQGDKKGKKGKKDRDMDELKKEVSMDDHKLSLDELHRKYGTDLSRGLTSARAAEILARDGPNALTPPPTTPEWIKFCRQLFGGFSMLLWIGAILCFLAYSIQAATEEEPQNDNLYLGVVLSAVVIITGCFSYYQEAKSSKIMESFKNMVPQQALVIRNGEKMSINAEEVVVGDLVEVKGGDRIPADLRIISANGCKVDNSSLTGESEPQTRSPDFTNENPLETRNIAFFSTNCVEGTARGIVVYTGDRTVMGRIATLASGLEGGQTPIAAEIEHFIHIITGVAVFLGVSFFILSLILEYTWLEAVIFLIGIIVANVPEGLLATVTVCLTLTAKRMARKNCLVKNLEAVETLGSTSTICSDKTGTLTQNRMTVAHMWFDNQIHEADTTENQSGVSFDKTSATWLALSRIAGLCNRAVFQANQENLPILKRAVAGDASESALLKCIELCCGSVKEMRERYAKIVEIPFNSTNKYQLSIHKNPNTSEPQHLLVMKGAPERILDRCSSILLHGKEQPLDEELKDAFQNAYLELGGLGERVLGFCHLFLPDEQFPEGFQFDTDDVNFPIDNLCFVGLISMIDPPRAAVPDAVGKCRSAGIKVIMVTGDHPITAKAIAKGVGIISEGNETVEDIAARLNIPVSQVNPRDAKACVVHGSDLKDMTSEQLDDILKYHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLIFIIANIPLPLGTVTILCIDLGTDMVPAISLAYEQAESDIMKRQPRNPKTDKLVNERLISMAYGQIGMIQALGGFFTYFVILAENGFLPIHLLGLRVDWDDRWINDVEDSYGQQWTYEQRKIVEFTCHTAFFVSIVVVQWADLVICKTRRNSVFQQGMKNKILIFGLFEETALAAFLSYCPGMGVALRMYPLKPTWWFCAFPYSLLIFVYDEVRKLIIRRRPGGWVEKETYY

SEQ ID NO: 2 - protein, 1013 aa, Homo sapiens
MGDKKDDKDSPKKNKGKERRDLDDLKKEVAMTEHKMSVEEVCRKYNTDCVQGLTHSKAQEILARDGPNALTPPPTTPEWVKFCRQLFGGFSILLWIGAILCFLAYGIQAGTEDDPSGDNLYLGIVLAAVVIITGCFSYYQEAKSSKIMESFKNMVPQQALVIREGEKMQVNAEEVVVGDLVEIKGGDRVPADLRIISAHGCKVDNSSLTGESEPQTRSPDCTHDNPLETRNITFFSTNCVEGTARGVVVATGDRTVMGRIATLASGLEVGKTPIAIEIEHFIQLITGVAVFLGVSFFILSLILGYTWLEAVIFLIGIIVANVPEGLLATVTVCLTLTAKRMARKNCLVKNLEAVETLGSTSTICSDKTGTLTQNRMTVAHMWFDNQIHEADTTEDQSGTSFDKSSHTWVALSHIAGLCNRAVFKGGQDNIPVLKRDVAGDASESALLKCIELSSGSVKLMRERNKKVAEIPFNSTNKYQLSIHETEDPNDNRYLLVMKGAPERILDRCSTILLQGKEQPLDEEMKEAFQNAYLELGGLGERVLGFCHYYLPEEQFPKGFAFDCDDVNFTTDNLCFVGLMSMIDPPRAAVPDAVGKCRSAGIKVIMVTGDHPITAKAIAKGVGIISEGNETVEDIAARLNIPVSQVNPRDAKACVIHGTDLKDFTSEQIDEILQNHTEIVFARTSPQQKLIIVEGCQRQGAIVAVTGDGVNDSPALKKADIGVAMGIAGSDVSKQAADMILLDDNFASIVTGVEEGRLIFDNLKKSIAYTLTSNIPEITPFLLFIMANIPLPLGTITILCIDLGTDMVPAISLAYEAAESDIMKRQPRNPRTDKLVNERLISMAYGQIGMIQALGGFFSYFVILAENGFLPGNLVGIRLNWDDRTVNDLEDSYGQQWTYEQRKVVEFTCHTAFFVSIVVVQWADLIICKTRRNSVFQQGMKNKILIFGLFEETALAAFLSYCPGMDVALRMYPLKPSWWFCAFPYSFLIFVYDEIRKLILRRNPGGWVEKETYY

SEQ ID NO: 3 - RNA, 19 nt, Artificial
  Small interfering RNA (si RNA) against the alpha1 subunit of human NA+/K+ ATPase (sense)
gggcaguguuucaggcuaa

SEQ ID NO: 4 - RNA, 19 nt, Artificial
  Small interfering RNA (si RNA) against the alpha1 subunit of human NA+/K+ ATPase (antisense)
uuagccugaaacacugccc

SEQ ID NO: 5 - RNA, 19 nt, Artificial
  Scrambled si RNA, control for si RNA against the alpha1 subunit of human NA+/K+ ATPase (sense)
ucuacgaggcacgagacuu

SEQ ID NO: 6 - RNA, 19 nt, Artificial
  Scrambled si RNA, control for si RNA against the alpha1 subunit of human NA+/K+ ATPase (antisense)
aacucucgugccucguaga

(19) United States
(12) Patent Application Publication
(10) Pub. No.: US 2010/0068198 A1
(43) Pub. Date: Mar. 18, 2010

(54) TARGETING OF ALPHA-1 OR ALPHA-3 SUBUNIT OF NA+, K+-ATPASE IN THE TREATMENT OF PROLIFERATIVE DISEASES

(75) Inventors: Tatjana Mijatovic, Rhode St Genese (BE); Eric Van Quaquebeke, Brussels (BE); Nancy Denève, Lahamaide (BE); Laurent Van den Hove, Forest (BE); Francis Darro, Uccle (BE); Robert Kiss, St-Pieters-Leeuw (BE)

Correspondence Address: KNOBBE MARTENS OLSON & BEAR LLP, 2040 MAIN STREET, FOURTEENTH FLOOR, IRVINE, CA, 92614, US

(73) Assignee: Unibioscreen S.A., Brussels (BE)

(21) Appl. No.: 12/513,663

(22) PCT Filed: Nov. 9, 2006
(86) PCT No.: PCT/EP2006/010739

§ 371 Date: Nov. 23, 2009

(51) Int. Cl. A61K 39/395; C07H 21/00; C07K 16/18; A61K 31/7088; A61P 35/00

(52) U.S. Cl. 424/130.1; 536/24.5; 530/387.1; 514/44 A

(57) ABSTRACT

Targeting of select subunits of the Na⁺,K⁺-ATPase is described, and especially the alpha-1 subunit and/or the alpha-3 subunit. Antisense agents, agents capable of causing RNA interference that can reduce the expression of Na⁺,K⁺-ATPase, and antibodies and antibody fragments and derivatives specific for Na⁺,K⁺-ATPase are useful as reagents and pharmaceutical formulations for the treatment of proliferative diseases, such as tumours and cancers. Methods and kits are also described.

### FIELD OF THE INVENTION

The present invention provides methods, reagents, pharmaceutical formulations and kits for the treatment of proliferative diseases, such as tumours and cancers. The invention teaches to target select subunits of the Na⁺,K⁺-ATPase.

### BACKGROUND OF THE INVENTION

The importance of devising novel and/or improved manners to combat proliferative disorders, such as diverse tumours and cancers, is self-evident. Hence, much research is invested into identifying cellular targets relevant in proliferative diseases, as well as designing therapeutic reagents that impinge on those targets.

The Na⁺,K⁺-ATPase (NKA or sodium pump) is an integral membrane protein found in the cells of all higher eukaryotes and transports Na⁺ and K⁺ ions across the plasma membrane using ATP hydrolysis (Horisberger 2004. Physiology (Bethesda) 19: 377-87; Lingrel and Kuntzweiler 1994. J Biol Chem 269: 19659-62). The sodium pump is composed of two subunits, alpha (α) and beta (β) in a substantially equimolar ratio. The alpha subunit is considered catalytic and includes binding sites for the Na⁺ and K⁺ ions. The beta subunit is regarded as regulatory and aiding the biogenesis and activity of the enzymatic complex. To date, four different alpha subunit isoforms (α1, α2, α3 and α4) and three distinct beta subunit isoforms (β1, β2, β3) have been identified in mammalian cells and they are selectively expressed in various tissues (Blanco 2005. Semin Nephrol 25: 292-303). Pestov et al. 1999 (FEBS Lett 456: 243-248, 1999) reported the presence of the fourth beta subunit (β-m) specifically found in muscles, but although it still remains unknown, however, whether this isoform forms part of the Na⁺,K⁺-ATPase or is a component of another X,K⁺-ATPase.

In addition to transporting ions, the sodium pump appears to interact with other cellular proteins, such as its neighbouring membrane proteins, and to participate in cytosolic signalling cascades. Several signalling pathways independent of changes in the intracellular Na⁺ and K⁺ concentrations have been reported to be activated or elicited by the interaction of cardiac glycosides, e.g., ouabain, with the sodium pump, including the activation of Src kinase, transactivation of the epidermal growth factor receptor by Src, activation of Ras and p42/p44 mitogen-activated protein kinases and increased generation of reactive oxygen species by mitochondria (Xie and Askari 2002. Eur J Biochem 269: 2434-2439; Wang et al. 2004. J Biol Chem 279: 17250-17259). There may even exist substantially distinct “pools” of sodium pumps within the plasma membrane of cells having different functions: one pool being (primarily) involved with the ion-transport while another pool (mainly) participating in signal transduction pathways (Xie and Askari 2002).

Cardiotonic steroids (CS) encompass a group of compounds that share the capacity to bind to the extracellular surface of the sodium pump, the binding site being composed of multiple functional groups in the alpha subunit and to a lesser extent in the beta subunit. Members of this group of compounds include plant-derived pharmaceuticals such as, e.g., the digitalis steroid glycoside drugs (digitoxin, digoxin, etc.) or the more polar plant monoglycoside, ouabain, and also vertebrate-derived aglycone CS such as bufalin and marinobufagenin. The classic actions of CS relate to their ability to inhibit the sodium pump, thereby increasing the intracellular sodium concentration. Accordingly, cardiac glycosides such as, e.g., digitoxin or ouabain, are commonly employed to treat congestive heart failure. However, new evidence also suggests CS actions that occur in the absence of substantial erosion of the transmembrane sodium gradient and that operate through intracellular second messenger signalling pathways with ultimate effects on gene expression and cell growth and division (Xie and Askari 2002).

Several cardiac glycosides (digitoxin, digoxin, ouabain and oleandrin, amongst others) have been shown to display anti-proliferative effects against human cancer cell lines in vitro (Xie and Cai 2003. Mol Intery 3: 157-68). For example, ouabain or digitalis cardiac glycosides were reported as apoptosis inducers in cellular models of glioblastoma (Haux 1999. Med Hypo 53: 543-548), prostate (McConkey et al. 2000. Cancer Res 60: 3807-3812) or breast cancers (Kometiani et al. 2005. Mol Pharmacol 67: 929-36).

Moreover, beneficial effects of digitalis treatment in breast cancer patients have been suggested by epidemiological studies. Stenkvist 2001 (Anticancer Drugs 12: 635-636) reported that the tumour cell populations from breast cancer patients on digitalis medication (for cardiac problems) were characterized by a number of cytometric features which strongly indicated that they had a lower proliferative capacity than tumour cells from patients not on digitalis treatment. Also, Stenkvist et al. 1982 (N Engl J Med 306: 484) found in a 5-year follow-up study that the recurrence rate among patients not on digitalis was 9.6 times higher than among patients on digitalis and after a 20-year follow-up (Stenkvist et al. 1999. Oncol Rep 6: 493-6) that the death rate from breast carcinoma was significantly lower (6%) among patients on digitalis compared with patients not on digitalis (34%).

The foregoing evidence suggests that the sodium pump is a relevant molecular target for the prevention and treatment of proliferative diseases. However, the battery of substances useful to impinge on this molecule is presently somewhat restricted.

Consequently, there exists a need for further reagents which target the sodium pump and, in particular, for such reagents having advantageous properties, such as, for example, reagents that are increasingly effective and/or decrease unwanted side-effects, and/or are more selective for cancerous cells, and/or are less erosive for healthy cells, and/or are comparably specific for particular cancer types, and/or are less toxic, etc. By means of example, the above exemplary or further improved property or properties of such reagents may be manifested vis-à-vis one or more substances known in the art to target the sodium pump, for example, one or more cardiac glycosides, such as, without limitation, one or more of ouabain, digitoxin or digoxin.

### SUMMARY OF THE INVENTION

In aspects, the present invention provides uses, methods, assays, reagents, compositions and kits that address at least some, e.g., one or more, of the above discussed needs of the art.

More specifically, the present invention surprisingly realised that when the alpha-1 (α1) subunit and/or the alpha-3 (α3) subunit of the Na⁺,K⁺-ATPase is targeted by agents that (a) reduce the expression of the said α1 and/or α3 subunits, or (b) bind to the said α1 and/or α3 subunits, such targeting displays one or more of the above discussed advantages in the framework of therapy of proliferative diseases.

By means of example and not limitation, such specific targeting of the α1 and/or α3 subunits of the sodium pump may provide for, e.g., increased efficacy, and/or less side-effects and/or increased selectivity towards the cancerous cells compared to healthy tissues.

Given the existence of at least 4 documented isoforms of alpha and three beta subunits, and consequently at least 12 different isoforms of the (alpha-N)₂(beta-N)₂ sodium pump, where N=1 to 4, it is very surprising that exactly the specific targeting of the alpha-1 and/or alpha-3 subunits should show particular benefits.

The present invention integrates the above relevant realisation in its diverse aspects.

Hence, in an aspect the invention concerns an agent that (a) can reduce the expression of the alpha-1 subunit of Na⁺,K⁺-ATPase or (b) can bind to the alpha-1 subunit of Na⁺,K⁺-ATPase, for use as a medicament, particularly in the treatment of a proliferative disorder.

In another aspect the invention concerns an agent that (a) can reduce the expression of the to alpha-3 subunit of Na⁺,K⁺-ATPase or (b) can bind to the alpha-3 subunit of Na⁺,K⁺-ATPase, for use as a medicament, particularly in the treatment of a proliferative disorder.

In a further aspect the invention relates to the use of an agent that (a) can reduce the expression of the alpha-1 subunit of Na⁺,K⁺-ATPase or (b) can bind to the alpha-1 subunit of Na⁺,K⁺-ATPase, for the preparation of a medicament for the treatment of a proliferative disorder.

In a further aspect the invention relates to the use of an agent that (a) can reduce the expression of the alpha-3 subunit of Na⁺,K⁺-ATPase or (b) can bind to the alpha-3 subunit of Na⁺,K⁺-ATPase, for the preparation of a medicament for the treatment of a proliferative disorder.

In a yet further aspect the invention concerns the use of an agent that (a) can reduce the expression of the alpha-1 subunit of Na⁺,K⁺-ATPase or (b) can bind to the alpha-1 subunit of Na⁺,K⁺-ATPase, and of an agent that (c) can reduce the expression of the alpha-3 subunit of Na⁺,K⁺-ATPase or (d) can bind to the alpha-3 subunit of Na⁺,K⁺-ATPase, for the preparation of a medicament for the treatment of a proliferative disorder.

In an aspect the invention provides a method for treating a proliferative disorder in a subject needing said therapy, comprising administering to the said subject a therapeutically effective amount of an agent that (a) can reduce the expression of the alpha-1 subunit of Na⁺,K⁺-ATPase or (b) can bind to the alpha-1 subunit of Na⁺,K⁺-ATPase.

In a further aspect the invention relates to a method for treating a proliferative disorder in a subject needing said therapy, comprising administering to the said subject a therapeutically effective amount of an agent that (a) can reduce the expression of the alpha-3 subunit of Na⁺,K⁺-ATPase or (b) can bind to the alpha-3 subunit of Na⁺,K⁺-ATPase.

In a yet another aspect the invention concerns a method for treating a proliferative disorder in a subject needing said therapy, comprising administering to the said subject a therapeutically effective amount of an agent that (a) can reduce the expression of the alpha-1 subunit of Na⁺,K⁺-ATPase or (b) can bind to the alpha-1 subunit of Na⁺,K⁺-ATPase, and of an agent that (c) can reduce the expression of the alpha-3 subunit of Na⁺,K⁺-ATPase or (d) can bind to the alpha-3 subunit of Na⁺,K⁺-ATPase,

In a related aspect, the invention provides a method comprising: (1) identifying or generating a first agent that (a) can reduce the expression of the alpha-1 subunit of Na⁺,K⁺-ATPase or (b) can bind to the alpha-1 subunit of Na⁺,K⁺-ATPase, and/or identifying or generating a second agent that (c) can reduce the expression of the alpha-3 subunit of Na⁺,K⁺-ATPase or (d) can bind to the alpha-3 subunit of Na⁺,K⁺-ATPase; and (2) using the first agent and/or the second agent for the preparation of a medicament for the treatment of a proliferative disorder.

In another related aspect, the invention discloses a method for treating a proliferative disorder in a subject needing said therapy, comprising: (1) identifying or generating a first agent that (a) can reduce the expression of the alpha-1 subunit of Na⁺,K⁺-ATPase or (b) can bind to the alpha-1 subunit of Na⁺,K⁺-ATPase, and/or identifying or generating a second agent that (c) can reduce the expression of the alpha-3 subunit of Na⁺,K⁺-ATPase or (d) can bind to the alpha-3 subunit of Na⁺,K⁺-ATPase; and (2) administering to the said subject a therapeutically effective amount of the first and/or the second agent.

In a further aspect, the invention provides a pharmaceutical composition comprising a therapeutically effective amount of an agent that (a) can reduce the expression of the alpha-1 subunit of Na⁺,K⁺-ATPase or (b) can bind to the alpha-1 subunit of Na⁺,K⁺-ATPase, an comprising a therapeutically effective amount of an agent that (c) can reduce the expression of the alpha-3 subunit of Na⁺,K⁺-ATPase or (d) can bind to the alpha-3 subunit of Na⁺,K⁺-ATPase, or a pharmaceutically acceptable salt of any such agent(s). Said pharmaceutical composition may commonly also comprise one or more of pharmaceutically acceptable buffers, carriers, excipients, stabilisers, etc.

In a further aspect, the invention provides kits comprising the above antibody agent(s) or pharmaceutical composition(s) alongside other reagent(s), composition(s) or device(s) generally useful in the treatment of proliferative diseases.

In still further aspect, the invention provides an assay to select, from a group of test agents, a candidate agent potentially useful as a therapeutic in the treatment of a proliferative disorder, said assay comprising determining whether a tested agent (a) can reduce the expression of the alpha-1 subunit of Na⁺,K⁺-ATPase or (b) can bind to the alpha-1 subunit of Na⁺,K⁺-ATPase, and/or (c) can reduce the expression of the alpha-3 subunit of Na⁺,K⁺-ATPase or (d) can bind to the alpha-3 subunit of Na⁺,K⁺-ATPase.

The said assay may further comprise monitoring the effect, e.g., therapeutic effect, of the so-selected candidate agent when administered to an in vitro or in vivo model of the proliferative disorder, e.g., a cellular, tissue or organism model, e.g., a non-human animal model, preferably a non-human mammal model. Otherwise, the said assay may comprise use of the so-selected candidate agent for the preparation of a composition for administration to and monitoring the effect, e.g., therapeutic effect, in a non-human animal model, preferably a non-human mammal model, of the proliferative disorder.

The invention also concerns the ensuing particularly preferred, yet exemplary and non-limiting embodiments of the above aspects.

In an embodiment, an agent that can reduce the expression of the alpha-1 and/or alpha-3 subunits of Na⁺,K⁺-ATPase is an antisense agent, e.g., an antisense oligonucleotide, or a ribozyme, or an agent capable of causing RNA interference.

In further embodiments, an agent that can bind to the alpha-1 and/or alpha-3 subunits of Na⁺,K⁺-ATPase is a polypeptide or protein, an antibody, a peptide, a peptidomimetic, an aptamer, a chemical substance (preferably an organic molecule, more preferably a small organic molecule), a lipid, a carbohydrate, a nucleic acid, etc.

In other embodiments, an agent that can specifically bind to the alpha-1 and/or alpha-3 subunits of Na⁺,K⁺-ATPase is also capable of altering, e.g., inhibiting or activating, one or more facets of the biological activity of Na⁺,K⁺-ATPase.

In further embodiment, the proliferative disorder is one that overexpresses the alpha-1 subunit and/or the alpha-3 subunit of the NKA.

In preferred embodiments, the proliferative disorder, e.g., one which overexpresses the alpha-1 and/or alpha-3 subunit of the NKA, is chosen from glioma, preferably glioblastoma; prostate cancer; non-small-cell lung cancer (NSCLC); or colon cancer.

In a particularly preferred embodiment, the proliferative disorder, especially one which overexpresses the alpha-1 subunit of the NKA, is NSCLC. The overexpression of the alpha-1 subunit of NKA in NSCLC is surprising as the said subunit has been reported to be downregulated in cancers (Sakai et al. 2004. FEBS Lett 563(1-3): 151-4).

These and further aspects and preferred embodiments of the invention are described in the following sections and in the appended claims.

### BRIEF DESCRIPTION OF FIGURES

FIG. 1 illustrates exemplary sequence of alpha-1 subunit of NKA.

FIG. 2 illustrates exemplary sequence of alpha-3 subunit of NKA.

FIG. 3 illustrates typical patterns of expression of the Na⁺/K⁺-ATPase α1, α2 and α3 subunits in normal lung parenchyma and bronchial tissues vis-à-vis NSCLC-ADCs and NSCLC-SCCs.

FIG. 4 illustrates more detailed quantitation of the Na⁺/K⁺-ATPase α1, α2 and α3 subunits in normal lung parenchyma and bronchial tissues, NSCLC-ADCs, NSCLC-SCCs, and cell lines.

FIG. 5 illustrates effects of Na⁺/K⁺-ATPase al depletion by siRNA.

FIG. 6 illustrates the in vitro anti-tumour effect of Compound 2, as compared to ouabain, digitoxin and digoxin.

### DETAILED DESCRIPTION OF THE INVENTION

As used herein, the singular forms “a”, “an”, and “the” include both singular and plural referents unless the context clearly dictates otherwise. By way of example, “an antibody” refers to one or more than one antibody; “an antigen” refers to one or more than one antigen.

The terms “comprising”, “comprises” and “comprised of” as used herein are synonymous with “including”, “includes” or “containing”, “contains”, and are inclusive or open-ended and do not exclude additional, non-recited members, elements or method steps.

The term “about” as used herein when referring to a measurable value such as a parameter, an amount, a temporal duration, and the like, is meant to encompass variations of ±20% or less, preferably ±10% or less, more preferably ±5% or less, even more preferably ±1% or less, and still more preferably ±0.1% or less from the specified value, insofar such variations are appropriate to perform in the disclosed invention.

All documents cited in the present specification are hereby incorporated by reference in their entirety. In particular, the teachings of all documents herein specifically referred to are incorporated by reference.

Hence, aspects of the invention concern agents that can reduce the expression of the alpha-1 subunit and/or of the alpha-3 subunit of Na⁺,K⁺-ATPase, and agents that can bind to the alpha-1 subunit and/or to the alpha-3 subunit of Na⁺,K⁺-ATPase, and the use of such agents in therapy, especially in the treatment of proliferative disorders, as set out in the Summary section.

As used herein, the term “agent” broadly refers to any chemical (e.g., inorganic or organic), biochemical or biological substance, molecule or macromolecule (e.g., biological macromolecule), a combination or mixture thereof, a sample of undetermined composition, or an extract made from biological materials such as bacteria, plants, fungi, or animal cells or tissues. Preferred though non-limiting “agents” include nucleic acids, oligonucleotides, ribozymes, polypeptides or proteins, a peptides, peptidomimetics, antibodies and fragments and derivatives thereof, aptamers, chemical substances, preferably organic molecules, more preferably small organic molecules, lipids, carbohydrates, polysaccharides, etc., and any combinations thereof.

The terms “polypeptide” and “protein” are used interchangeably herein and generally refer to a polymer of amino acid residues linked by peptide bonds, and are not limited to a minimum length of the product. Thus, peptides, oligopeptides, polypeptides, dimers (hetero- and homo-), multimers (hetero- and homo-), and the like, are included within the definition. Both full-length proteins and fragments thereof are encompassed by the definition. The terms also include post-expression modifications of the polypeptide, for example, glycosylation, acetylation, phosphorylation, etc. Furthermore, for purposes of the present invention, the terms also refer to such when including modifications, such as deletions, additions and substitutions (e.g., conservative in nature), to the sequence of a native protein or polypeptide.

The term “peptide” as used herein preferably refers to a polypeptide as used herein consisting essentially of ≦50 amino acids, e.g., ≦45 amino acids, preferably ≦40 amino acids, e.g., ≦35 amino acids, more preferably ≦30 consecutive amino acids, e.g., ≦25, ≦20, ≦15, ≦10 or ≦5 amino acids.

The term “nucleic acid” as used herein means a polymer of any length composed essentially of nucleotides, e.g., deoxyribonucleotides and/or ribonucleotides. Nucleic acids can comprise purine and/or pyrimidine bases, and/or other natural, chemically or biochemically modified (e.g., methylated), non-natural, or derivatised nucleotide bases. The backbone of nucleic acids can comprise sugars and phosphate groups, as can typically be found in RNA or DNA, and/or one or more modified or substituted (such as, 2′-O-alkylated, e.g., 2′-O-methylated or 2′-O-ethylated; or 2′-O,4′-C-alkynelated, e.g., 2′-O,4′-C-ethylated) sugars or one or more modified or substituted phosphate groups. For example, backbone analogues in nucleic acids may include phosphodiester, phosphorothioate, phosphorodithioate, methylphosphonate, phosphoramidate, alkyl phosphotriester, sulfamate, 3′-thioacetal, methylene (methylimino), 3′-N-carbamate, morpholino carbamate, and peptide nucleic acids (PNAs).

The term “nucleic acid” further specifically encompasses DNA, RNA and DNA/RNA hybrid molecules, specifically including hnRNA, pre-mRNA, mRNA, cDNA, genomic DNA, gene, amplification products, oligonucleotides, and synthetic (e.g. chemically synthesised) DNA, RNA or DNA/RNA hybrids. The terms “ribonucleic acid” and “RNA” as used herein mean a polymer of any length composed of ribonucleotides. The terms “deoxyribonucleic acid” and “DNA” as used herein mean a polymer of any length composed of deoxyribonucleotides. The term “DNA/RNA hybrid” as used herein mean a polymer of any length composed of one or more deoxyribonucleotides and one or more ribonucleotides.

A nucleic acid can be naturally occurring, e.g., present in or isolated from nature, can be recombinant, i.e., produced by recombinant DNA technology, and/or can be, partly or entirely, chemically or biochemically synthesised. A nucleic acid can be double-stranded, partly double stranded, or single-stranded. Where single-stranded, the nucleic acid can be the sense strand or the antisense strand. In addition, nucleic acid can be circular or linear.

The term “oligonucleotide” as used herein denotes single stranded nucleic acids (nucleotide multimers) of greater than 2 nucleotides in length and preferably up to 200 nucleotides in length, more preferably from about 10 to about 100 nucleotides in length, even more preferably from about 12 to about 50 nucleotides in length. Oligonucleotides can be synthesised by any method known in the art, e.g., by chemical or biochemical synthesis, e.g., solid phase phosphoramidite chemical synthesis, or by in vitro or in vivo expression from recombinant nucleic acid molecules, e.g., bacterial or retroviral vectors.

The term “can”, as in “can reduce expression” or “can specifically bind to”, is synonymous to “is capable of” and signifies that an entity, e.g., an agent, has the ability to achieve the recited effect or action, e.g., when administered to a patient or to a relevant in vitro or in vivo model system, as opposed to achieving the recited effect or action at the exact time of the recitation (which may but need not be the case).

The terms “Na⁺,K⁺-ATPase”, “NKA”, “sodium pump” and variants thereof refer herein to integral membrane proteins commonly known under such designations in the art, which are found in higher eukaryotes and transport Na⁺ and K⁺ ions across the plasma membrane using ATP hydrolysis (for a review see Horisberger 2004; Lingrel and Kuntzweiler 1994; supra). NKA is also known as EC 3.6.3.9. These terms encompass such proteins from any organism where found, and particularly from animals, preferably vertebrates, more preferably mammals, including humans and non-human mammals. The terms as used herein refer to Na⁺,K⁺-ATPase when forming part of a living organism, organ, tissue, and/or cell, as well as when at least partly isolated therefrom, reconstituted, etc. The terms also encompass Na⁺,K⁺-ATPase when one, more or all of its parts have been expressed using recombinant DNA technology.

A prototypic structural organisation of NKA comprises a hetero-tetramer composed of two alpha (α) and two beta (β) subunits. The present invention particularly concerns specific isoforms of the alpha subunit, i.e., isoforms alpha-1 (α1) and alpha-3 (α3).

As used herein, the terms “alpha-1” or “α1” subunit and “alpha-3” or “α3” subunit refer to respective subunits of the Na⁺,K⁺-ATPase commonly known under such designations in the art. The terms encompass such subunits from any organism where found, and particularly from animals, preferably vertebrates, more preferably mammals, including humans and non-human mammals.

An “alpha-1”/“α1” subunit or “alpha-3”/“α3” subunit as used herein refer to polypeptides with a “native” sequence, i.e., polypeptides of which the primary sequence is the same as that of an NKA alpha-1 or alpha-3 subunit, respectively, derived from nature. A skilled person understands that the native sequence of α1 subunit, or that of the α3 subunit, may differ between different species due to genetic divergence between such species. Moreover, the native sequence of α1 subunit, or that of the α3 subunit, may differ between or even within different individuals of the same species due to normal genetic diversity (variation) within a given species. Also, the native sequence of α1 subunit, or that of the α3 subunit, may differ between or even within different individuals of the same species due to post-transcriptional modifications, e.g., differential splicing, RNA editing, etc. Accordingly, all alpha-1 or alpha-3 sequences found in nature, and preferably those defining biologically functional molecules, are considered native.

The “alpha-1”/“α1” subunit or “alpha-3”/“α3” subunit as used herein may form part of a living organism, organ, tissue, and/or cell, or may be (at least partly) isolated therefrom, reconstituted, etc. The terms also encompass the respective subunits when produced by recombinant or synthetic means.

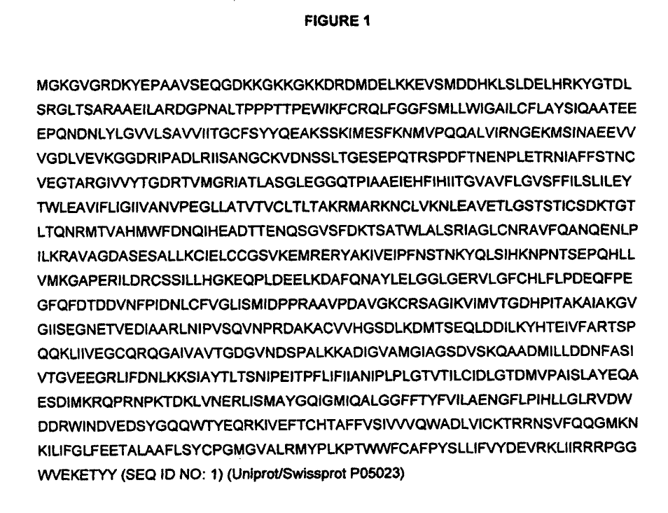

Exemplary NKA alpha-1 subunits include, without limitation, human α1 subunit with primary sequence as annotated under Uniprot/Swissprot (http://www.expasy.org/) accession number P05023 (also shown in FIG. 1 as SEQ ID NO: 1), as well as α1 subunits from other animals having primary sequences as annotated in the same database, e.g., from dog (acc. no. P50997), pig (P05024), sheep (P04074), horse (P18907), chicken (P09572), mouse (Q8VDN2) or rat (P06685). A skilled person will appreciate that some of the above sequences may include precursor pro-peptides that may be (at least partly) absent from the mature proteins. For example, the Uniprot/Swissprot entry for human α1 subunit (P05023) specifies a pro-peptide composed of amino acids 1-5 as shown in SEQ ID NO: 1. Similar pro-peptides may be present in other α1 subunit precursors. As noted, intra-species sequence variation, or post-translational modifications, etc., can produce other native alpha-1 subunit sequences that differ to some extent from those listed above.

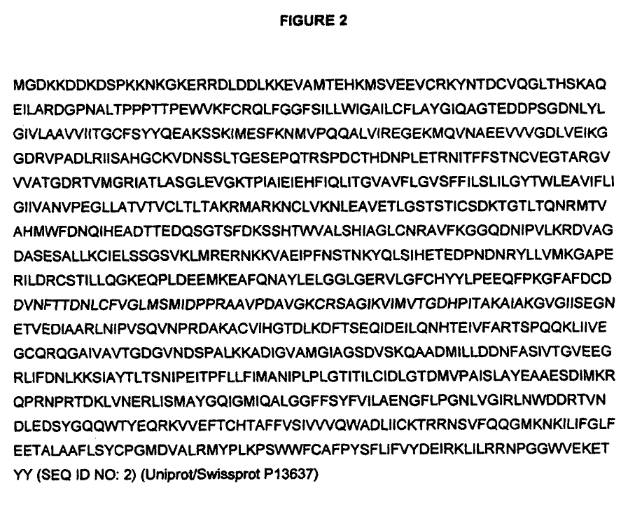

Exemplary NKA alpha-3 subunits include, without limitation, human α3 subunit with primary sequence as annotated under Uniprot/Swissprot accession number P13637 (also shown in FIG. 2 as SEQ ID NO: 2), as well as α1 subunits from other animals having primary sequences as annotated in the same database, e.g., from chicken (P24798), mouse (Q6PIC6) or rat (P06687). A skilled person will appreciate that the above or other α3 sequences might include precursor pro-peptides that may be absent from the mature proteins. As noted, intra-species sequence variation, or post-translational modifications, etc., can produce other native alpha-3 subunit sequences that differ to some extent from those listed above.

The term “isolated” refers to a molecule which has been identified and separated and/or recovered from a component of its natural environment. For instance, an isolated protein can be substantially separated from cellular material or other proteins from the cell or tissue source from which it is derived. Similarly, an isolated nucleic acid can be substantially separated from cellular material or other nucleic acids from the cell or tissue source from which it is derived.

The term “isolated” also refers to preparations where the isolated molecule, e.g., a polypeptide or protein, or a nucleic acid, is substantially pure, e.g., at least 70-80% pure by weight, more preferably at least 80-90% pure by weight, even more preferably at least 90-95% pure by weight, and most preferably at least 95%, 96%, 97%, 98%, 99%, or 100% pure by weight. For example, purity of a polypeptide or protein may be determined by the Lowry method. Also, an isolated polypeptide or protein may be purified to homogeneity as determined by SDS-PAGE under reducing or non-reducing conditions using Coomassie blue or, preferably, silver stain. Yet alternatively, an isolated polypeptide or protein may be purified to a degree sufficient to obtain at least 15 residues of N-terminal or internal amino acid sequence by use of a spinning cup sequenator. For example, purity of a nucleic acid may be determined by measuring absorbance A₂₆₀/A₂₈₀. Also, an isolated nucleic acid may be purified to homogeneity as determined by agarose- or polyacrylamide-gel electrophoresis and ethidium bromide or similar staining.

Agents Binding to the Alpha-1 Subunit and/or to the Alpha-3 Subunit of Na⁺,K⁺-ATPase

Hence, in an aspect of the invention agents can bind to the alpha-1 subunit and/or to the alpha-3 subunit of Na⁺,K⁺-ATPase.

The term “binding” as used herein generally refers to a physical association, preferably herein a non-covalent physical association, between molecular entities, e.g., between a “ligand” (generally referring to any agent, e.g., a substance or molecule) and a “receptor” (generally referring to any molecule). Preferably, a “receptor” may be a polypeptide or protein, such as, e.g., the alpha-1 subunit or the alpha-3 subunit of NKA, or variants or fragments thereof, or a nucleic acid encoding such, etc. Preferably, a “ligand” may be, e.g., a polypeptide or protein, an antibody, a peptide, a peptidomimetic, an aptamer, a chemical substance (preferably an organic molecule, more preferably a small organic molecule), a lipid, a carbohydrate, a nucleic acid, etc.

In preferred embodiments, an agent is capable of binding to native conformation of the alpha-1 subunit and/or of the alpha-3 subunit of NKA.

The term “native conformation” is used to refer to a conformation substantially retaining the secondary and tertiary structure of the native state of a protein. By means of example, the alpha-1 subunit or the alpha-3 subunit of NKA are said to have native conformation if they to substantially retain the secondary and tertiary structure of the respective native subunits, preferably when making a part of a native NKA, e.g., preferably enzymatically active NKA.

Advantageously, an agent binding the native conformation of a target polypeptide is awaited to be particularly effective in vivo where the respective target protein, e.g., a specific NKA alpha subunit, is expected to be mainly found in its native, or substantially native, conformation.

In embodiments, an agent can bind to (1) the extracellular portion of, or (2) the intracellular portion of, or (3) the transmembrane portion of the alpha-1 subunit of NKA or of the alpha-3 subunit of NKA, or (concurrently) to two or more of the said portions of the respective subunits.

The term “extracellular portion” of the alpha-1 subunit or of the alpha-3 subunit of NKA, as used herein, refers to those portions of the respective subunits which are normally, and preferably when the respective subunits are in the native conformation, e.g., when the respective subunits make a part of a native NKA, e.g., preferably enzymatically active. NKA, exposed toward the outer space of a cell or toward the lumen of intracellular membrane-bound organelles. The complementary term “intracellular portion” then denotes those portions of the alpha-1 subunit or of the alpha-3 subunit of NKA which face the cytoplasm of the cell. The term “transmembrane portion” consequently designates those portions of the alpha-1 subunit or of the alpha-3 subunit of NKA which are embedded within the cellular membranes.

By means of example and not limitation, the Uniprot/Swissprot entry P05023 for human alpha-1 subunit indicates that amino acids 109-131, 309-320, 793-802, 867-918 and 971-985 of the alpha-1 sequence as shown in SEQ ID NO: 1 are particularly predicted as constituting or contributing to the lumenal/extracellular portion of this subunit; amino acids 6-87, 153-288, 339-772, 824-843, 939-951 and 1007-1023 of the alpha-1 sequence as shown in SEQ ID NO: 1 are particularly predicted as constituting or contributing to the cytoplasmic portion of this subunit; and amino acids 88-108, 132-152, 289-308, 321-338, 773-792, 803-823, 844-866, 919-938, 952-970 and 98-1006 of the alpha-1 sequence as shown in SEQ ID NO: 1 are particularly predicted as constituting or contributing to the transmembrane portion of this subunit.

Also by means of example and not limitation, the Uniprot/Swissprot entry P13637 for human to alpha-3 subunit indicates that amino acids 99-121, 299-310, 783-792, 857-908 and 961-975 of the alpha-3 sequence as shown in SEQ ID NO: 2 are particularly predicted as constituting or contributing to the lumenal/extracellular portion of this subunit; amino acids 1-77, 143-278, 329-762, 814-833, 929-941 and 997-1013 of the alpha-3 sequence as shown in SEQ ID NO: 2 are particularly predicted as constituting or contributing to the cytoplasmic portion of this subunit; and amino acids 78-98, 122-142, 279-298, 311-328, 763-782, 793-813, 834-856, 909-928, 942-960 and 976-996 of the alpha-3 sequence as shown in SEQ ID NO: 2 are particularly predicted as constituting or contributing to the transmembrane portion of this subunit.

A skilled person is aware of algorithms for predicting the membrane topology of polypeptides or proteins, as well as of experimental methods for verifying the so-predicted topology, and could apply such methods to predict and/or determine the lumenal/extracellular, transmembrane and intracellular/cytoplasmic regions of the alpha-1 subunit or of the alpha-3 subunit of NKA. For example, suitable prediction algorithms for this purpose are described in Bernsel and Von Heijne 2005 (Protein Science 14: 1723-1728).

In a preferred embodiment, an agent can bind to the extracellular portion of alpha-1 and/or alpha-3 subunit. For example, such binding does not require the agent to cross the cell membrane in order to effect the binding, thereby potentially simplifying the delivery of the agent.

In preferred embodiments, an agent is capable of binding to the alpha-1 subunit and/or of the alpha-3 subunit of NKA under physiological conditions. “Physiological conditions” are those conditions characteristic of an organism's (e.g., a subject's to-be-treated, e.g., an animal or human subject's) healthy or normal functioning.

In preferred embodiments of the aspects of the invention, an agent of the invention binds to the alpha-1 subunit and/or to the alpha-3 subunit of NKA with high affinity.

As used herein, binding can be considered “high affinity” when the affinity constant (K_{A}) of such binding is K_{A}≧1×10⁴ M⁻¹, preferably K_{A}≧1×10⁵ M⁻¹, even more preferably K_{A}≧1×10⁶ M⁻¹ such as, e.g., K_{A}≧1×10⁷ M⁻¹, yet more preferably K_{A}≧1×10⁸ M⁻¹, even more preferably K_{A}≧1×10⁹ M⁻¹, e.g., K_{A}≧1×10¹⁰ M⁻¹, and most preferably K_{A}≧1×10¹¹ M⁻¹, e.g., K_{A}≧1×10¹² M⁻¹, K_{A}≧1×10¹³ M⁻¹, K_{A}≧1×10¹⁴ M⁻¹, K_{A}≧1×10¹⁵ M⁻¹ or even higher, wherein K_{A}=[Ligand_Receptor]/[Ligand][Receptor]. Determination of K_{A} can be carried out by methods known in the art, such as, e.g., using equilibrium dialysis and Scatchard plot analysis.

Advantageously, high-affinity binding allows to reduce the quantity of an agent required to achieve a therapeutic effect in a patient, owing to the comparably high strength of interaction between the agent and its molecular target.

In further preferred embodiments of the aspects of the invention, binding of an agent of the invention to the alpha-1 subunit and/or to the alpha-3 subunit of NKA can be specific.

The term “specifically bind” or “specific binding” reflects a situation when a ligand binds to a given receptor more readily than it would bind to a random, unrelated receptor. For example, a ligand (agent) specifically binding to a polypeptide or protein (1) preferably displays little or no binding to other polypeptides, and preferably to homologues or orthologues of the polypeptide or protein (1), under conditions where it would specifically bind the said polypeptide or protein (1). Under little or no binding is meant K_{A}≦1×10⁴ M⁻¹, preferably K_{A}≦1×10³ M⁻¹, more preferably K_{A}≦1×10² M⁻¹, yet more preferably K_{A}≦1×10¹ M⁻¹, e.g., K_{A}≦1 M⁻¹, most preferably K_{A}<<1 M⁻¹, e.g., K_{A}≦1×10⁻¹ M⁻¹, K_{A}≦1×10⁻² M⁻¹, K_{A}≦1×10⁻³ M⁻¹, K_{A}≦1×10⁻⁴ M⁻¹, K_{A}≦1×10⁻⁵ M⁻¹, K_{A}≦1×10⁻⁶ M⁻¹, or smaller.

By means of example and not limitation, an agent specifically binding to the alpha-1 subunit of NKA preferably shows little or no binding to any other NKA alpha subunit isoforms, such as alpha-2, alpha-3 and alpha-4. An agent specifically binding to the alpha-3 subunit of NKA preferably shows little or no binding to any other NKA alpha subunit isoforms, such as alpha-1, alpha-2 and alpha-4. An agent specifically binding to the alpha-1 and the alpha-3 subunits of NKA preferably shows little or no binding to any other NKA alpha subunit isoforms, such as alpha-2 and alpha-4.

Advantageously, such specific binding reduces the potential effects of agents on receptors other than their specific target, including effects on NKA molecules other than those comprising the specifically targeted alpha subunit, thereby improving the selectivity of the treatment and reducing the chance of unwanted side-effects.

An agent that binds, preferably with high affinity and specifically, to the alpha-1 and/or the alpha-3 subunit of NKA may, in preferred embodiments, also alter, e.g., inhibit or activate, the biological activity of NKA, i.e., may be an NKA “inhibitor” or “activator”.

When such agent is said to be an NKA “inhibitor” or “activator”, this generally means that binding of the said agent to one or both alpha subunits of NKA will reduce or increase, respectively, one or more aspects of the said NKA biological activity than if the said agent had not been bound thereto. These terms may also refer to that administration of the said agent to an in vitro system, cell, tissue or an organism comprising NKA biological activity, preferably to a patient, will reduce or increase, respectively, one or more aspects of the said NKA biological activity than if the said agent had not been administered.

An aspect of NKA biological activity is the enzymatic activity thereof, i.e., the capacity for ATP hydrolysis-driven exchange of Na⁺ and K⁺ ions across membranes; accordingly, an NKA “inhibitor” may inhibit the enzymatic activity of NKA, and an NKA “activator” may activate the enzymatic activity of NKA. For example, an exemplary way of measuring/testing the level, inhibition or activation of the enzymatic activity of NKA by an agent of interest is shown in example 3.

An aspect of NKA biological activity is control of signalling pathways, e.g., pathways involving Src kinase, epidermal growth factor receptor, Ras, p42/p44 mitogen-activated protein kinases and increased generation of reactive oxygen species (Xie and Askari 2002; Wang et al. 2004; supra); accordingly, an NKA “inhibitor” may inhibit one or more of the NKA-controlled signalling pathways, and an NKA “activator” may activate one or more of the NKA-controlled signalling pathways. A skilled person will appreciate that a given ligand may impinge, also differently, on more aspects of the biological activity of NKA, e.g., on both above mentioned aspects. By means of example and not limitation, a given ligand may inhibit the enzymatic activity of NKA but activate one or more NKA-controlled signalling pathways. Hence, an agent referred to as an NKA “inhibitor”, e.g., for its effect on NKA enzymatic activity, may in fact activate one or more of the NKA-controlled signalling pathways. Or a given agent may inhibit one or more NKA-controlled signalling pathways but activate one or more other NKA-controlled signalling pathways, etc.

The terms “inhibit” and “activate” encompass any extents of, respectively, inhibition or activation. For example, inhibition of one or more (independently) aspects of NKA biological activity, e.g., its enzymatic and/or signalling activity, may be by at least about 10%, e.g., by at least about 20%, preferably by at least about 30%, e.g., by at least about 40%, more preferably by at least about 50%, e.g., by at least about 60%, even more preferably by at least about 70%, e.g., by at least about 80%, and most preferably by at least about 90%, e.g., by at least about 95%, such as by at least about 96%, 97%, 98%, 99% or even by 100%, when an agent is bound to one or both alpha subunits of NKA.

For example, activation of one or more (independently) aspects of NKA biological activity, e.g., its enzymatic and/or signalling activity, may be by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, when an agent is bound to one or both alpha subunits of NKA.

In preferred embodiments, an agent that binds to the alpha-1 and/or alpha-3 subunit of NKA may elicit one, more than one or all of the following effects: 1. inhibit the enzymatic activity of NKA; 2. reduce cellular expression of caveolin-1 (see, e.g., Glenney et al. 1992. FEBS Lett 314: 45-48 and Swissprot Q03135 for description of human caveolin-1); 3. cause disorganisation of cellular actin cytoskeleton; 4. causes depletion of cellular ATP; 5. cause dissociation of interaction between the NKA alpha subunit and cellular actin cytoskeleton. The present inventors realised that one or more of the above effects might be particularly relevant for the treatment of proliferative diseases according to the invention. The above effects of agents can be examined in suitable model systems, e.g., cellular or non-human animal model systems, e.g., as illustrated in examples 3 or 4, or using methods known in the art, e.g., immunocytochemistry, confocal microscopy and/or immunoprecipitation.

In a further preferred embodiment, an agent that can bind to the alpha-1 subunit and/or alpha-3 subunit of NKA may elicit anti-proliferation and/or anti-migratory effect in cell culture and/or non-human animal models of relevant proliferative diseases, e.g., as shown in example 6.

In preferred embodiments, an agent or ligand capable of binding to the alpha-1 subunit and/or to the alpha-3 subunit of NKA can be chosen from the group consisting of a chemical substance, preferably an organic molecule, more preferably a small organic molecule; a peptide; a peptidomimetic; a polypeptide or protein; an antibody, including fragments and derivatives thereof; an aptamer; a lipid; a carbohydrate; or a nucleic acid, including an oligonucleotide. Such agents may be isolated or substantially isolated as defined herein.

Many of the above recited types of agents, e.g., chemical substances, peptides, aptamers, carbohydrates, or nucleic acids, are available in synthetic, combinatorial and natural product libraries, and can be selected therefrom using screening assays of the invention determining binding of test agents to the alpha-1 and/or alpha-3 subunits of NKA.

In a preferred embodiment, an agent or ligand capable of binding to the alpha-1 subunit and/or to the alpha-3 subunit of NKA is a chemical substance, preferably an organic molecule, more preferably a small organic molecule.

The terms “chemical substance” or “chemical compound” as used herein refer to their connotation in the art; the terms encompass substances consisting of two or more different chemically bonded chemical elements, with a fixed ratio determining the composition. The term includes both inorganic and organic compounds.

The terms “organic compound” or “organic molecule” as used herein refer to their broad connotation in the art. The terms encompass organic molecules which are natural products, as well as which are semi- or fully synthesised.

The term “small organic molecule”, as used herein, refers to organic compounds with a size comparable to those organic molecules generally used in pharmaceuticals. The term excludes biological macromolecules (e.g., proteins, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da.

In an embodiment, the organic molecule is selected from the group comprising or consisting of a compound of formula I,

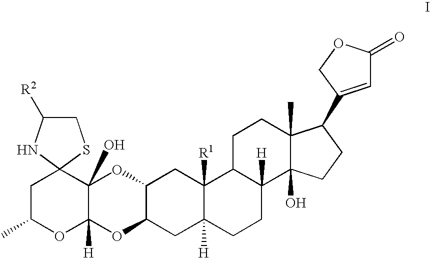

wherein R¹ is selected from formyl, hydroxyC₁₋₄alkyl, C₁₋₄alkylcarbonyloxyC₁₋₄alkyl, C₅₋₁₂arylcarbonyloxyC₁₋₄alkyl, R² is selected from oxo, C₁₋₄alkylcarbonylC₁₋₄alkyl, C₅₋₁₂arylcarbonylC₁₋₄alkyl, or R² is a double bond between the carbon atom bearing R² and the N atom of the heterocyclic ring. Preferably, R¹ is selected from formyl, hydroxymethyl, hydroxyethyl, methylcarbonyloxymethyl, ethylcarbonyloxymethyl, propylcarbonyloxymethyl, phenylcarbonyloxymethyl, R² is selected from oxo, methylcarbonyloxymethyl, ethylcarbonyloxymethyl, propylcarbonyloxymethyl, phenylcarbonyloxymethyl, or R² is a double bond between the carbon atom bearing R² and the N atom of the heterocyclic ring.

Without limitation, exemplary compounds of this embodiment include those of Table 1:

TABLE 1

| Compound | R¹ | R² |
|---|---|---|
| 1 | —COH | ═O |
| 2 | —CH₂OH | ═O |
| 3 | —CH₂OAc | ═O |
| 4 | —CH₂OOCphenyl | ═O |
| 5 | —CH₂OH | Double bond^{*} |
| 6 | —CH₂OAc | Double bond^{*} |
| 7 | —CH₂OOCphenyl | Double bond^{*} |
| 8 | —CH₂OH | 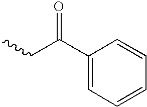 |
| 9 | —CH₂OH | 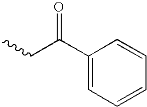 |

means a double bond between the N atom and the C carbon atom of the N-containing heterocyclic ring of formula I.

In a further preferred embodiment, the organic molecule is the compound 2 of the following formula:

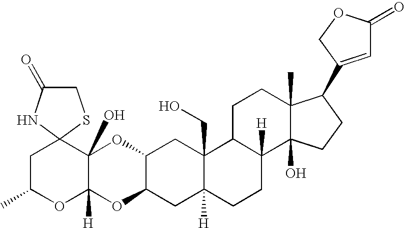

Compounds as above can be (hemi)synthesized using methods detailed, e.g., in Van Quaquebeke et al. 2005 (J Med Chem 48: 849-856), or modifications thereof.

In a further preferred embodiment, an agent or ligand capable of binding to the alpha-1 subunit and/or to the alpha-3 subunit of NKA is a peptidomimetic, esp. a peptidomimetic of a peptide that binds to the respective subunit(s).

As used herein, the term “peptidomimetic” refers to a non-peptide agent that is a topological analogue of a corresponding peptide. Methods of rationally designing peptidomimetics of peptides are known in the art. For example, the rational design of three peptidomimetics based on the sulphated 8-mer peptide CCK26-33, and of two peptidomimetics based on the 11-mer peptide Substance P, and related peptidomimetic design principles, are described in Horwell 1995 (Trends Biotechnol 13: 132-134).

Peptidomimetics often show improved properties, e.g., improved stability, greater resistance to hydrolysis, or easier delivery, than their corresponding peptides.

In a further preferred embodiment, an agent or ligand capable of binding to the alpha-1 subunit and/or to the alpha-3 subunit of NKA is an aptamer.

The term “aptamer” as used herein refers to single-stranded or double-stranded oligo-DNA, oligo-RNA or oligo-DNA/RNA or any analogue thereof, that specifically bind to and alter the biological activity of a target molecule, preferably of a polypeptide or protein, such as, e.g., the alpha-1 subunit or the alpa-3 subunit of NKA. Aptamers are capable of binding their respective targets under physiological conditions. Selection of aptamers in vitro allows rapid isolation of extremely rare oligos that have high specificity and affinity for specific proteins. Exemplary RNA aptamers are described in U.S. Pat. No. 5,270,163, Ellington and Szostak 1990 (Nature 346: 818-822), Tuerk and Gold 1990 (Science 249: 505-510), incorporated by reference herein. RNA aptamers can frequently discriminate finely among discrete functional sites of a protein, see Gold et al. 1995 (Annu Rev Biochem 64: 763-797).

In a preferred embodiment, an agent or ligand capable of binding to the alpha-1 subunit and/or to the alpha-3 subunit of NKA is an antibody, including fragments and derivatives thereof.

As used herein, the term “antibody” is used in its broadest sense and generally refers to any immunologic binding agent. The term specifically encompasses intact monoclonal antibodies, polyclonal antibodies, multivalent (e.g., 2-, 3- or more-valent) and/or multi-specific antibodies (e.g., bi- or more-specific antibodies) formed from at least two intact antibodies, and antibody fragments insofar they exhibit the desired biological activity (particularly, ability to specifically bind an antigen of interest), as well as multivalent and/or multi-specific composites of such fragments. The term “antibody” is not only inclusive of antibodies generated by methods comprising immunisation, but also includes any polypeptide, e.g., a recombinantly expressed polypeptide, which is made to encompass at least one complementarity-determining region (CDR) capable of specifically binding to an epitope on an antigen of interest. Hence, the term applies to such molecules regardless whether they are produced in vitro or in vivo.

Preferably, the antibody for use in the methods of the invention may be isolated. An “isolated” antibody is one which has been identified and separated and/or recovered from a component of its natural environment. Contaminant components of its natural environment are materials that would interfere with therapeutic uses for the antibody, and may include enzymes, hormones, other proteinaceous or non-proteinaceous solutes, etc. Preferably, an isolated antibody is purified (1) to greater than 80% by weight of antibody as determined by the Lowry method, more preferably to greater than 90% by weight, even more preferably to greater than 95% by weight and most preferably to greater than 99% by weight; and/or (2) to homogeneity by SDS-PAGE under reducing or non-reducing conditions using Coomassie blue or, preferably, silver stain; and/or (3) to a degree sufficient to obtain at least 15 residues of N-terminal or internal amino acid sequence by use of a spinning cup sequenator. Isolated antibody includes the antibody in situ within recombinant cells since at least one component of the antibody's natural environment will not be present. Ordinarily, however, isolated antibody will be prepared by at least one purification step.

The present antibody can thus preferably “specifically bind to” or is “specific for” the alpha-1 subunit and/or the alpha-3 subunit of NKA (which therefore is the antibody's antigen), meaning that the antibody can bind to an epitope of the respective subunit through its complementarity determining region (CDR), and that the said binding entails some complementarity between the CDR and the epitope. Specific binding between an antibody and an antigen is normally non-covalent and reversible. Hence, an antibody “specifically binds” or is “specific for” the respective subunit when it binds to an epitope of that subunit via its CDR more readily than it would bind to a random, unrelated epitope.

Specific binding is usually signified by high affinity and low to moderate capacity, whereas non-specific binding mostly has low affinity and moderate to high capacity. As used herein, the term “affinity” of an antibody toward an antigen refers to a measure of the strength of the binding of an individual epitope with the CDR of an antibody molecule. See, e.g., Harlow et al. 1988. Antibodies: A Laboratory Manual, Cold Spring Harbour Laboratory Press, 2nd ed., p. 27-28. The term “avidity” then refers to the overall stability of the complex between a population of immunoglobulins and an antigen, that is, the functional combining strength of an immunoglobulin mixture with the antigen. See, e.g., Harlow at p. 29-34.

As used herein, binding of an antibody to an antigen is considered specific when the affinity constant K_{A}≧1×10⁴ M⁻¹, preferably K_{A}≧1×10⁵ M⁻¹, even more preferably K_{A}≧1×10⁶ M⁻¹ such as, e.g., K_{A}≧1×10⁷ M⁻¹, yet more preferably K_{A}≧1×10⁸ M⁻¹, e.g., K_{A}≧1×10⁹ M⁻¹, K_{A}≧1×10¹⁰ M⁻¹, and most preferably K_{A}≧1×10¹¹ M⁻¹, e.g., K_{A}≧1×10¹² M⁻¹, K_{A}≧1×10¹³ M⁻¹, K_{A}≧1×10¹⁴ M⁻¹, K_{A}≧1×10¹⁵ M⁻¹ or even higher, wherein K_{A}=[AgAb]/[Ag][Ab]. The binding affinity of an antibody can, for example, be determined by the Scatchard plot analysis of Munson et al. 1980 (Anal Biochem 107: 220), e.g., as in the BIAcore system (Biacore AB, Uppsala, Sweden).

Antibodies may also be described in terms of their cross-reactivity. As used herein, the term “cross-reactivity” refers to the ability of an antibody specific for one antigen to also react with a second antigen; i.e., a measure of relatedness between two different antigenic substances. Thus, an antibody is cross-reactive if it binds to an epitope other than the one that induced its formation. The cross-reactive epitope can generally contain many of the same complementary structural features as the inducing epitope.

An antibody may be said to have little or no “cross-reactivity” if it, under conditions where it would specifically bind its inducing (i.e., specific) epitope, does not substantially bind (e.g., K_{A}≦1×10⁴ M⁻¹, preferably K_{A}≦1×10³ M⁻¹, more preferably K_{A}≦×10² M⁻¹, yet more preferably K_{A}≦1×10¹ M⁻¹, e.g., K_{A}≦1 M⁻¹, most preferably K_{A}<<1 M⁻¹, e.g., K_{A}≦1×10⁻¹ M⁻¹, K_{A}≦1×10⁻² M⁻¹, K_{A}≦1×10⁻³ M⁻¹, K_{A}≦1×10⁻⁴ M⁻¹, K_{A}≦1×10⁻⁵ M⁻¹, K_{A}≦1×10⁻⁶ M⁻¹, or smaller) other epitopes, such as, e.g., epitopes with less than 99.5%, less than 99%, less than 98%, less than 97%, less than 96%, less than 95%, less than 94%, less than 93%, less than 92%, less than 91%, less than 90%, less than 85%, less than 80%, less than 75%, less than 70%, less than 65%, less than 60%, less than 55%, and/or less than 50% sequence identity to its specific epitope.

An antibody specific to a given polypeptide or protein may be said to have little or no “cross-reactivity” with homologues or orthologues of the said polypeptide or protein if it, under conditions where it would specifically bind the said polypeptide or protein, does not substantially bind (e.g., K_{A}≦1×10⁴ M⁻¹, preferably K_{A}≦1×10³ M⁻¹, more preferably K_{A}≦1×10² M⁻¹, yet more preferably K_{A}≦1×10¹ M⁻¹, e.g., K_{A}≦1 M⁻¹, most preferably K_{A}<<1 M⁻¹, e.g., K_{A}≦1×10⁻¹ M⁻¹, K_{A}≦1×10⁻² M⁻¹, K_{A}≦1×10⁻³ M⁻¹, K_{A}≦1×10⁻⁴ M⁻¹, K_{A}≦1×10⁻⁵ M⁻¹, K_{A}≦1×10⁻⁶ M⁻¹, or smaller) such homologues or orthologues.

For example, an antibody specific to a given polypeptide or protein (1) may be said to have little or no “cross-reactivity” with other polypeptides or proteins (2), e.g., with homologues or orthologues of the said polypeptide or protein (1), when the extent of binding of the antibody to such polypeptides or proteins (2) will be less than 10%, preferably less than 5%, even more preferably less than 1%, yet more preferably less than 0.1%, most preferably less than 0.01% or even less than 0.001%, of the total binding of the antibody to polypeptides or proteins (1) and (2), as determined by, e.g., fluorescence activated cell sorting (FACS) analysis or (radio)immunoprecipitation (RIA). Exemplarily, this can also apply to the above discussed NKA alpha subunits.

In preferred embodiments, an antibody specific for the alpha-1 subunit of NKA can preferably show little or no cross-reactivity with any other NKA alpha subunit isoforms, such as alpha-2, alpha-3 and alpha-4. An antibody specific for the alpha-3 subunit of NKA can preferably show little or no cross-reactivity with any other NKA alpha subunit isoforms, such as alpha-1, alpha-2 and alpha-4. An antibody specific for the alpha-1 and the alpha-3 subunits of NKA can preferably show little or no cross-reactivity with any other NKA alpha subunit isoforms, such as alpha-2 and alpha-4.

In an embodiment, the antibody may be an intact antibody.

An “intact” antibody is one which comprises an antigen-binding variable region as well as a light chain constant domain (CL) and heavy chain constant domains, CH1, CH2 and CH3. The constant domains may be native sequence constant domains (e.g. human native sequence constant domains) or amino acid sequence variant thereof.

Depending on the amino acid sequence of the constant domain of their heavy chains, intact antibodies can be assigned to different “classes”, contemplated in the invention. There are five major classes of intact antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The “light chains” of antibodies from vertebrate species can be assigned to one of two clearly distinct types, called kappa (κ) and lambda (λ), based on the amino acid sequences of their constant domains. The subunit structures and three-dimensional configurations of different classes of immunoglobulins are well known.

In an embodiment, the antibody may be any of the above Ig classes, and preferably IgG class antibody.

Most native vertebrate, incl. mammalian antibodies are usually heterotetrameric glycoproteins of about 150,000 daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has substantially regularly spaced intrachain disulfide bridges. Each heavy chain has at one end a variable domain (VH) followed by a number of constant domains. Each light chain has a variable domain at one end (VL) and a constant domain at its other end. The constant domain of the light chain is aligned with the first constant domain of the heavy chain, and the light-chain variable domain is aligned with the variable domain of the heavy chain. Particular amino acid residues are believed to form an interface between the light chain and heavy chain variable domains.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called hypervariable regions both in the light chain and the heavy chain variable domains. The more highly conserved portions of variable domains are called the framework regions (FRs). The variable domains of native heavy and light chains usually each comprise four FRs, connected by three hypervariable regions. The hypervariable regions in each chain are held together in close proximity by the FRs and, with the hypervariable regions from the other chain, contribute to the formation of the “antigen-binding site” of antibodies (see Kabat et al. 1991. Sequences of Proteins of Immunological Interest, 5th Ed. Public Health Service, National Institutes of Health, Bethesda, Md.). The constant domains are not involved directly in binding an antibody to an antigen, but exhibit various effector functions, such as participation of the antibody in antibody dependent cellular cytotoxicity (ADCC).

In some instances, e.g., certain immunoglobulin molecules derived from camelid species or engineered based on camelid immunoglobulins, a complete immunoglobulin molecule may consist of heavy chains only, with no light chains (see, e.g., Hamers-Casterman et al. 1993. Nature 363: 446-448). Hence, in these immunoglobulins the heavy chain variable region, referred to as VHH, forms the entire CDR. These molecules and functional fragments and/or derivatives thereof are also included by the term “antibody” as used herein. Accordingly, in an embodiment, the antibody may be a camelid antibody as described above.

In a preferred embodiment, the antibody is a monoclonal antibody or a mixture of monoclonal antibodies. Monoclonal antibodies offer the advantages of, e.g., selectively and reproducibly targeting a particular antigen and even a particular epitope within the said antigen, as well as reproducible production and titre, amongst others evident to a skilled person.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations that include different antibodies directed against different antigenic determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method.

By means of example and not limitation, monoclonal antibodies to be used in accordance with the present invention may be made by the hybridoma method first described by Kohler et al. 1975 (Nature 256: 495), or may be made by recombinant DNA methods (e.g., as in U.S. Pat. No. 4,816,567). Monoclonal antibodies may also be isolated from phage antibody libraries using techniques as described by Clackson et al. 1991 (Nature 352: 624-628) and Marks et al. 1991 (J Mol Biol 222: 581-597), for example.

The monoclonal antibodies as defined herein also specifically include “chimeric” antibodies in is which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (see, e.g., U.S. Pat. No. 4,816,567; Morrison et al. 1984. PNAS 81: 6851-6855). Accordingly, in an embodiment the antibody may be a chimeric antibody.

Exemplary chimeric antibodies of interest herein include “primatised” antibodies comprising variable domain antigen-binding sequences derived from a non-human primate (e.g. Old World Monkey, Ape etc.) and human constant region sequences.

“Humanised” forms of non-human (e.g., rodent) antibodies are chimeric antibodies that contain minimal sequence derived from non-human immunoglobulin. For the most part, humanised antibodies are human immunoglobulins (recipient antibody) in which residues from a hypervariable region of the recipient are replaced by residues from a hypervariable region of a non-human species (donor antibody) such as, e.g., mouse, rat, rabbit or nonhuman primate, having the desired specificity, affinity, and capacity. In some instances, framework region (FR) residues of the human immunoglobulin are replaced by corresponding non-human residues. Furthermore, humanised antibodies may comprise residues that are not found in the recipient antibody or in the donor antibody. These modifications are made to further refine antibody performance. In general, the humanised antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanised antibody optionally also will comprise at least a portion of an immunoglobulin constant region (Fc), typically that of a human immunoglobulin. For further details on performing humanised antibodies, see, for example, Jones et al. 1986 (Nature 321: 522-525), Riechmann et al. 1988 (Nature 332: 323-329) and Presta 1992 (Curr Op Struct Biol 2: 593-596). Accordingly, in an embodiment the antibody may be a humanised antibody, e.g., to advantageously minimise the immune response against the non-human portions of the original antibody.

The term “hypervariable region” when used herein refers to the amino acid residues of an antibody which are responsible for antigen-binding. The hypervariable region generally comprises amino acid residues from a “complementarity determining region” or “CDR” (e.g. residues 24-34 (L1), 50-56 (L2) and 89-97 (L3) in the light chain variable domain and 31-35 (H1), 50-65 (H2) and 95-102 (H3) in the heavy chain variable domain; Kabat et al. 1991. Sequences of Proteins of Immunological Interest, 5th Ed. Public Health Service, National Institutes of Health, Bethesda, Md.) and/or those residues from a “hypervariable loop” (e.g., residues 26-32 (L1), 50-52 (L2) and 91-96 (L3) in the light chain variable domain and 26-32 (H1), 53-55 (H2) and 96-101 (H3) in the heavy chain variable domain; Chothia and Lesk 1987. J Mol Biol 196: 901-917). “Framework Region” or “FR” residues are those variable domain residues other than the hypervariable region residues.

In further embodiments, the antibody agent may be antibody fragments as described here below. Some advantages of such fragments include, e.g., smaller size, easier delivery, absence of effector domains, etc.

“Antibody fragments” comprise a portion of an intact antibody, comprising the antigen-binding or variable region thereof. Examples of antibody fragments include Fab, Fab′, F(ab′)2, Fv and scFv fragments; diabodies; linear antibodies; single-chain antibody molecules; and multivalent and/or multispecific antibodies formed from antibody fragment(s), e.g., dibodies, tribodies, and multibodies. The above designations Fab, Fab′, F(ab′)2, Fv, scFv etc. are intended to have their art-established meaning.

By means of further explanation, papain digestion of antibodies produces two identical antigen-binding fragments, called “Fab” fragments, each with one antigen-binding site, and a residual “Fc” fragment. Pepsin treatment yields an F(ab′)2 fragment that has two antigen-binding sites. Typical Fc fragment comprises the C-terminal portions of both H chains bound by disulfides. The effector functions of antibodies are determined by sequences in the Fc region, which region is also the part recognized by Fc receptors (FcR) found on certain types of cells.

“Fv” is an antibody fragment which contains a complete antigen-recognition and antigen-binding site. This region consists essentially of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain, VH or VL, i.e., half of an Fv comprising only three hypervariable regions specific for an antigen, has the ability to recognize and bind antigen, although at a lower affinity than the entire binding site (“single-domain antibodies”).

“Single-chain Fv” or “scFv” antibody fragments comprise the VH and VL domains of antibody, wherein these domains are present in a single polypeptide chain. Preferably, the Fv polypeptide further comprises a polypeptide linker between the VH and VL domains which enables the scFv to form the desired structure for antigen binding. For a review of carrying out scFv see Plückthun in The Pharmacology of Monoclonal Antibodies, vol. 113, Rosenburg and Moore eds., Springer-Verlag, New York, pp. 269-315 (1994).

Moreover, the term “Fv” also encompasses further functional (i.e., specifically antigen-binding) fragments thereof. Examples of such fragments include but are not limited to a “minibody” which comprises a fragment of the heavy chain only of the Fv, a “microbody” which comprises a small fractional unit of antibody heavy chain variable region (see PCT/IL99/00581), similar bodies having a fragment of the light chain, and similar bodies having a functional unit of a light chain variable region. It shall be appreciated that a fragment of an Fv molecule can be a substantially circular or looped polypeptide.

A typical Fab fragment also contains the constant domain of the light chain and the first constant domain (CH1) of the heavy chain. Fab′ fragments differ from Fab fragments by the addition of a few residues at the C-terminus of the heavy chain CH1 domain including one or more Cys residues from the antibody hinge region. Fab′-SH is the designation herein for Fab′ in which the cysteine residue(s) of the constant domains bear at least one free thiol group.

“F(ab′)2” antibody fragments originally were produced as pairs of Fab′ fragments which have hinge Cys residues between them. Other chemical couplings of antibody fragments are also known and encompassed within the term.

The term “diabodies” refers to small antibody fragments with two antigen-binding sites, which fragments comprise a variable heavy domain (VH) connected to a variable light domain (VL) in the same polypeptide chain (VH-VL). By using a linker that is too short to allow pairing between the two domains on the same chain, the domains are forced to pair with the complementary domains of another chain and create two antigen-binding sites. Diabodies are described more fully in, for example, EP 404,097, WO 93/11161, and Hollinger et al. 1993 (PNAS 90: 6444-6448).

In a further embodiment, the antibody may be a polyclonal antibody, as defined below.

The term “polyclonal antibody” refers to antibodies that are heterogeneous populations of antibody molecules having antigen-binding functions specific for different epitopes, such as, e.g., for different epitopes of the same antigen. Typically, polyclonal antibodies may be derived from sera of animals immunised with an antigen. More specifically, the term also encompasses whole antisera, antibody populations representative of whole antisera, as well as subpopulations of antibodies from whole antisera, such as, e.g., Ig class-specific subpopulations or antigen-specific subpopulations (e.g., by affinity purification).

Preferably, the polyclonal antibody may be isolated away from the serum components and/or may be Ig class purified and/or affinity purified, thus leading to greater specificity and lower risk of non-specific reactions.

Apart from antigen-binding functions, some antibodies, such as particularly native antibodies, entail “effector functions” which refer to those biological activities attributable to the Fc region (a native sequence Fc region or a functional amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; down regulation of cell surface receptors (e.g. B cell receptor; BCR), etc.

A skilled person appreciates that effector function(s) of an antibody may be decreased or eliminated without substantially diminishing the ability of the antibody to bind its respective antigen. By means of example and not limitation, the Fc portion or a part thereof responsible for one or more effector functions to be eliminated, can be removed from an antibody. Alternatively, the Fc portion of an antibody may be mutated at one or more amino acid positions to decrease or eliminate its effector function(s). Moreover, if an effector domain of an antibody is effective in one species, it may be less effective or silent in another species.

Accordingly, in a preferred embodiment the antibody does not comprise effector functions, e.g., lacks regions responsible for such effector functions or contains variations which reduce or eliminate such effector functions, etc. Preferably, the antibodies so-lack (e.g., deletion, mutation, modification, etc.) regions that can entail effector functions in the organism to which the antibody is to be administered in the treatment methods of the invention. Advantageously, such antibodies will specifically bind to the respective alpha subunit(s) of the NKA, but will not induce reaction of the complement or immune systems (e.g., cellular or humoral) against cells which are bound by such antibodies. This can reduce the risk of unwanted effects of the treatment.

The term antibody includes antibodies originating from or comprising one or more portions derived from any animal species, preferably vertebrate species, including, e.g., birds and mammals. Without limitation, the antibodies may be chicken, turkey, goose, duck, guinea fowl, quail or pheasant. Also without limitation, the antibodies may be human, murine (e.g., mouse, rat, etc.), donkey, rabbit, goat, sheep, guinea pig, camel (e.g., Camelus bactrianus and Camelus dromaderius), llama (e.g., Lama paccos, Lama glama or Lama vicugna) or horse. Further without limitation, the variable region may be condricthoid in origin (e.g., from sharks).

As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described infra and, for example in, U.S. Pat. No. 5,939,598.

A skilled person will understand that an antibody can include one or more amino acid deletions, additions and/or substitutions (e.g., conservative substitutions), insofar such alterations preserve its binding of the respective antigen. An antibody may also include one or more native or artificial modifications of its constituent amino acid residues (e.g., glycosylation, etc.).

Methods of producing polyclonal and monoclonal antibodies as well as fragments thereof are well known in the art (see for example, Harlow and Lane, Antibodies: A Laboratory Manual, Cold Spring Harbour Laboratory, New York, 1988, incorporated herein by reference). As well as methods to produce recombinant antibodies or fragments thereof. For examples of methods of the preparation and uses of monoclonal antibodies, see, e.g., U.S. Pat. No. 5,688,681; U.S. Pat. No. 5,688,657; U.S. Pat. No. 5,683,693; U.S. Pat. No. 5,667,781; U.S. Pat. No. 5,665,356; U.S. Pat. No. 5,591,628; U.S. Pat. No. 5,510,241; U.S. Pat. No. 5,503,987; U.S. Pat. No. 5,501,988; U.S. Pat. No. 5,500,345 and U.S. Pat. No. 5,496,705; Skerra et al. 1993 (Curr Opinion in Immunol 5: 256-262); Plückthun 1992 (Immunol Revs 130: 151-188); McCafferty et al. 1990 (Nature 348: 552-554); Clackson et al. 1991 (Nature 352: 624-628); Marks et al. 1991 (J Mol Biol 222: 581-597); Marks et al. 1992 (BioTechnology 10: 779-783), Waterhouse et al. 1993 (Nuc Acids Res 21: 2265-2266); U.S. Pat. No. 4,816,567; Morrison et al. 1984 (PNAS 81: 6851); incorporated by reference in their entirety. Examples of the preparation and uses of polyclonal antibodies are disclosed in U.S. Pat. No. 5,512,282; U.S. Pat. No. 4,828,985; U.S. Pat. No. 5,225,331 and U.S. Pat. No. 5,124,147 which are incorporated by reference in their entirety. For examples of methods for preparation of antibody fragments, see, e.g., Morimoto et al. 1992 (J Biochem Biophys Methods 24: 107-117); Brennan et al. 1985 (Science 229: 81); Carter et al. 1992 (BioTechnology 10: 163-167); WO 93/16185; U.S. Pat. No. 5,571,894; U.S. Pat. No. 5,587,458; U.S. Pat. No. 5,641,870; incorporated by reference in their entirety. EP 0 656 946 describes the isolation and uses of camelid immunoglobulins and is incorporated herein by reference.

Typically, production of antibodies according to the invention may comprise immunisation of a host animal, preferably a vertebrate, more preferably a mammal, with a suitable antigen.

As used herein, the term “antigen” denotes any substance capable of eliciting an immune response in a host, and in particular capable of eliciting a humoral response involving the production of antibodies specific for the said antigen. An antigen comprises one or more than one antigenic determinants or epitopes which may be the same or different. The term “antigenic determinant” or “epitope” refers to a site of an antigen that is complementary to an antigen-binding site of a corresponding antibody and thus capable of specifically interacting with the latter.

For particular purposes of the invention, an “antigen” can comprise, consist essentially of, or consist of the alpha-1 or the alpha-3 subunit of the sodium pump, fragments thereof (e.g., including ≧4, ≧5, ≧6, ≧8, ≧10, preferably ≧15, more preferably ≧20, even more preferably ≧25, ≧30, ≧40, ≧50, ≧100 or ≧500 consecutive amino acids thereof; or, e.g., including ≧10%, ≧20%, ≧30%, ≧40%, ≧50%, ≧60%, ≧70%, ≧80% or ≧90% of the polypeptide sequence), variants thereof (e.g., including one or more amino acid deletions, additions and/or substitutions, preferably conservative substitutions, wherein sequence identity with the native protein or fragment thereof—e.g., as determined by NCBI BLAST sequence alignment algorithm—can be ≧50%, ≧60%, preferably ≧70%, more preferably ≧80%, even more preferably ≧90%. ≧95%, ≧99%), derivatives thereof (e.g., including derivations of one or more amino acid residues thereof, e.g., by glycosylation, phosphorylation, disulphide bridge, etc., wherein the fraction of modified amino acids vis-à-vis the native protein or fragment thereof or variant thereof can be ≧50%, ≧40%, ≧30%, preferably ≧20%, more preferably ≧10%, even more preferably ≧5%. e.g., ≧4%, ≧3%, ≧2% or ≧1%) or genetic or chemical fusions of any of the above with heterologous presenting carriers, e.g., GST, HBc, keyhole limpet hemocyanin, serum albumin, bovine thyroglobulin, or soybean trypsin inhibitor etc., insofar the above induce production of antibodies specific for (one or more epitopes of) the native alpha-1 or alpha-3 subunits. These modifications may be deliberate, as through site-directed mutagenesis, or may be accidental, such as through mutations of hosts that produce the antigens.

Alternatively, cells expressing the alpha-1 subunit and/or alpha-3 subunit at their cell surface can be used to generate antibodies. Other forms of alpha-1 and/or alpha-3 subunits useful for generating antibodies will be apparent to those skilled in the art.

Antigenic regions of proteins, esp. of the alpha-1 subunit and/or alpha-3 subunits of NKA, can be identified using, e.g., standard antigenicity and hydropathy plots as calculated, for instance, using Hopp/Woods method for antigenicity profiles (Hopp et al. 1981. PNAS 78: 3824-3828) and the Kyte-Doolittle technique for hydropathy plots (Kyte et al. 1982. J Mol Biol 157: 105-132). Such prediction programs are also included in standard sequence analysis software, e.g., in the GCG™ v. 11.1.2 package from Accelrys.

An envisaged epitope within a polypeptide or protein molecule can be “linear”, i.e., involving several consecutive amino acids, e.g., between about 5 and 12 adjacent amino acids, or between about 6 and 10 adjacent amino acids of the polypeptide or protein molecule. An envisaged epitope within a polypeptide or protein molecule can also be “conformational”, i.e., formed by amino acids that are not, or not all of which are, arranged sequentially in the primary amino acid sequence of the polypeptide or protein molecule, but which are so-juxtaposed within the 3-dimensional, folded structure of the native polypeptide or protein, as to be recognised by an antibody. An epitope may also involve further structural features of a native polypeptide or protein, such as, without limitation, glycosylation, phosphorylation, etc.

In a preferred embodiment, the epitope recognised by an antibody of the invention is in the extracellular portion of the alpha-1 subunit of NKA or of the alpha-3 subunit of NKA. This will facilitate access and binding of the administered antibody to the respective subunits. Accordingly, in an embodiment the immunisation antigen can comprise, consist essentially of or consist of at least a part of the extracellular portion of alpha-1 subunit or alpha-3 subunit of NKA, variant or derivative thereof, free or linked to a presenting carrier.

Antibodies generated against the alpha-1 subunit and/or alpha-3 subunit of NKA as inducing antigen can be tested for binding to the respective subunit(s) using methods well-known in the art, e.g., immunoprecipitation, affinity chromatography, ELISA, RIA, denaturing or non-denaturing immunoblotting, immunocytochemitry, immunohistochemistry, etc., such as to select antibodies having properties as above and useful in the methods of the invention. The binding affinity of the monoclonal antibody can, for example, be determined by the Scatchard analysis of Munson et al. 1980 (Anal Biochem 107: 220). Similarly, methods for isolation and purification of antibodies, e.g., affinity purification, protein A-Sepharose, hydroxylapatite chromatography, gel electrophoresis, dialysis, salt precipitation, etc., are well-known in the art.

Agents Reducing the Expression of the Alpha-1 and/or to the Alpha-3 Subunit NKA

In a further aspect of the invention agents can reduce the expression of the alpha-1 subunit and/or of the alpha-3 subunit of Na⁺,K⁺-ATPase.

When an agent, e.g., a substance or molecule, is said to “reduce the expression” of the alpha-1 subunit or of the alpha-3 subunit of NKA, this generally means that administration of the said substance to a cell, tissue or an organism, causes the respective subunits to be expressed at a level relatively lower than if the said substance had not been administered. Such reduction of expression can be observed and quantified, e.g., at the level of heterogeneous nuclear RNA (hnRNA), precursor mRNA (pre-mRNA), mRNA, cDNA and/or the protein of the respective subunits. Suitable methods to detect and quantify expression are known in the art and include, without limitation, Northern blotting, quantitative RT-PCR, Western blotting, ELISA, RIA, immunoprecipitation, etc. The term encompasses any extent of reduction of expression, such as, by way of example, reduction of expression by at least about 10%, e.g., at least about 20%, preferably at least about 30%, e.g., at least about 40%, more preferably at least about 50%, e.g., at least about 60%, even more preferably at least about 70%, e.g., at least about 80%, and most preferably at least about 90%, or even higher, e.g., at least about 95%, at least about 96%, at least about 97%, at least about 98%, at least about 99% or even (about) 100%, e.g., as measured in gross mass and/or at the level of individual cells.

In preferred embodiments, an agent or ligand capable of reducing the expression of the alpha-1 subunit and/or to the alpha-3 subunit of NKA can be chosen from the group consisting of a chemical substance, preferably an organic molecule, more preferably a small organic molecule; an antisense agent, e.g., an antisense oligonucleotide, a ribozyme, or an agent capable of causing RNA interference. Such agents may be isolated or substantially isolated as defined herein.

In a preferred embodiment, such agent specifically reduces the expression of the alpha-1 subunit and/or to the alpha-3 subunit of NKA, which it aims to target. The term “specifically reduces” reflects a situation when an agent reduces the expression of its target as above without substantially reducing the expression of another random, unrelated molecule.

In a preferred embodiment, an agent capable of reducing the expression of the alpha-1 subunit and/or to the alpha-3 subunit of NKA is an antisense reagent, esp. an antisense oligonucleotide.

The term “antisense” as used herein refers to a molecule designed to interfere with gene expression and capable of specifically binding to a desired target polynucleotide sequence. Antisense molecules typically (but not necessarily) comprise an oligonucleotide or oligonucleotide analogue capable of specifically hybridising to the target sequence. Hence, the term “antisense” oligonucleotide refers to an oligonucleotide or oligonucleotide analogue comprising, consisting essentially of or consisting of a nucleic acid sequence that is complementary or substantially complementary (i.e., largely but not wholly complementary) to a sequence within genomic DNA, hnRNA, mRNA or cDNA, preferably mRNA or cDNA, encoding a protein of interest; such as, e.g., within the genomic DNA, hnRNA, mRNA or cDNA, preferably mRNA or cDNA, of the alpha-1 subunit or the alpha-3 subunit of NKA. “Substantially complementary” refers to at least 85% complementary, e.g., preferably at least 90% complementary, e.g., at least 91% complementary, 92% complementary, more preferably at least 93% complementary, e.g., 94% complementary, even more preferably at least 95% complementary, e.g., at least 96% complementary, yet more preferably at least 97% complementary, e.g., at least 98% complementary, and most preferably at least 99% complementary. It is contemplated that antisense oligonucleotide may be complementary or substantially complementary to any of the 5′ untranslated region, the coding region and/or the 3′ untranslated region of an mRNA or cDNA.

Without being limited to any theory or mechanism, it is generally believed that the activity of antisense oligonucleotides depends on the binding of the oligonucleotide to the target nucleic acid, thus disrupting the function of the target, either by hybridization arrest (e.g., preventing the action of polymerases RNA processing) or by destruction of target RNA by RNase H (the ability to activate RNase H when hybridised to RNA) resulting in inhibition of expression.

In this and below references, the terms “hybridisation” or “hybridise” as used herein, refers to any process by which a strand of nucleic acid binds with a strand comprising complementary sequence(s) through base pairing, preferably involving hydrogen bonding, more preferably by Watson-Crick base pairing interactions. Hybridisation can take place between distinct strands or within the same strand.

Hybridisation and the strength of hybridisation (i.e., the strength of the association between the nucleic acid strands) is impacted by such factors as the degree of complementary between the nucleic acids, stringency of the conditions involved, the melting temperature of the formed hybrid, and the G:C (or U:C for RNA) ratio within the nucleic acids. In addition to sequence information, it is possible to determine if a nucleic acid has ≧85, ≧90, ≧95 or even ≧100% identity/complementarity by hybridisation at high stringency. “High stringency” conditions include conditions equivalent to the following exemplary conditions for binding or hybridisation at 65° C. in a solution consisting of 5×SSPE (43.8 g/l NaCl, 6.9 g/l NaH₂PO₄.H₂O and 1.85 g/l EDTA, pH adjusted to 7.4 with NaOH), 0.1% SDS, 5×Denhardt's reagent (50×Denhardt's contains per 500 ml: 5 g Ficoll (Type 400, Pharmacia), 5 g BSA (Fraction V; Sigma) and 100 μg/ml denatured salmon sperm DNA), followed by washing in a solution comprising 5×SSPE, 0.1% SDS at 65° C. when a probe of about 500 nucleotides in length is employed. Other exemplary conditions for hybridisation at “high stringency” for nucleic acid sequences over approximately 50-100 nucleotides in length include conditions equivalent to hybridisation in 6×SSC at 45° C., followed by one or more washes in 0.2×SSC, 0.1% SDS at 65° C. Numerous equivalent conditions may be employed to vary stringency conditions; factors such as the length and nature (DNA, RNA, base composition) of the probe and nature of the target (DNA, RNA, base composition, present in solution or immobilised, etc.) and the concentration of the salts and other components (e.g., the presence or absence of formamide, dextran sulphate, polyethylene glycol) are considered and the hybridisation solution may be varied to generate conditions of low or high stringency hybridisation different from, but equivalent to, the above listed conditions. In addition, the art knows conditions that promote hybridisation under conditions of high stringency (e.g., increasing the temperature of the hybridisation and/or wash steps, the use of formamide in the hybridisation solution, etc.). Guidance for performing hybridisation reactions can be found, for example, in Current Protocols in Molecular Biology, John Wiley & Sons, N.Y., 6.3.1-6.3.6, 1989, and more recent updated editions, all of which are incorporated by reference.

Typically, antisense agents suitable for the present invention may be capable of hybridising to their respective target at high stringency conditions. Such agents may hybridise specifically to the target under physiological conditions.

The terms “complementary” or “complementarity” as used herein with reference to nucleic acids, refer to the normal binding of polynucleotides under permissive salt (ionic strength) and temperature conditions by base pairing, preferably Watson-Crick base pairing. By means of example, complementary Watson-Crick base pairing occurs between the bases A and T, A and U or G and C. For example, the sequence A-G-T (i.e., 5′-A-G-T-3′) is thus complementary sequence T-C-A (i.e., 5′-T-C-A-3′).

Complementarity between two single-stranded nucleic acid molecules may be “partial”, such that only some nucleotides of the nucleic acids would bind when the strands hybridise, or it may be “complete”, such that total complementarity exists between the single stranded molecules. By means of example, a relatively shorter nucleic acid strand would show total complementarity to a relatively longer nucleic acid strand, if the latter strand comprised a sequence fully complementary to the sequence of the former strand.

The “degree of complementarity” of a nucleic acid molecule (1) to a nucleic molecule (2) can be expressed as the proportion (percentage) of nucleotides of the nucleic acid (1) molecule that would be expected to match, i.e., form Watson-Crick base-pairing, with nucleotides of the nucleic acid molecule (2), when the said nucleic acid molecules (1) and (2) were hybridised, preferably in high stringency conditions.

By “encoding” is meant that a nucleic acid sequence or its part corresponds, by virtue of the genetic code (of an organism in question, preferably mammalian, e.g., human), to a particular amino acid sequence, e.g., the amino acid sequence of a particular polypeptide or protein. By means of example, a nucleic acid sequence “encoding” a particular polypeptide or protein may include naturally-occurring genomic, hnRNA, pre-mRNA, mRNA (or therefrom obtained cDNA) for the said polypeptide or protein, or may include recombinant counterparts or variants of such naturally-occurring nucleic acid sequences.

By nucleic acid sequence encoding the alpha-1 subunit or the alpha-3 subunit of NKA, or any (preferably functional) variant or fragment thereof, is meant a nucleic acid sequence that corresponds, by virtue of the genetic code (of an organism in question, preferably mammalian, e.g., human), to the amino acid sequences of the said subunits, variants or fragments. By means of example and not limitation, a nucleic acid sequence encoding the alpha-1 subunit or the alpha-3 subunit of NKA may include the respective, native genomic, hnRNA, pre-mRNA, mRNA (or therefrom obtained cDNA) sequences for the said subunits, or may include recombinant counterparts or variants of such native nucleic acid sequences.

A skilled person understands that native nucleic acid sequences encoding the NKA α1 subunit, or the NKA α3 subunit, may differ between different species due to genetic divergence between such species. Moreover, the native nucleic acid sequences encoding the NKA α1 subunit, or the α3 subunit, may differ between or even within different individuals of the same species due to normal genetic diversity (variation) within a given species, or due to post-translational modifications. Accordingly, all nucleic acid sequences encoding alpha-1 or alpha-3 subunits found in nature, and preferably those encoding biologically functional polypeptide molecules, are considered native.

Exemplary cDNA sequences for NKA alpha-1 subunit include, without limitation, human α1 subunit cDNA sequence as annotated in the NCBI GenBank (http://www.ncbi.nlm.nih.gov/) under accession number NM_{—}000701. Exemplary cDNA sequences for NKA alpha-3 subunit include, without limitation, human α3 subunit cDNA sequence as annotated in the NCBI GenBank under accession number NM_{—}152296.

In a further preferred embodiment, an agent capable of reducing the expression of the alpha-1 subunit and/or to the alpha-3 subunit of NKA is a ribozyme.

The term “ribozyme” as used herein refers to a nucleic acid molecule, preferably an oligonucleotide or oligonucleotide analogue, capable of catalytically cleaving a polynucleotide. Preferably, a “ribozyme” may be capable of cleaving mRNA of a given polypeptide or protein, thereby reducing translation thereof; such as, preferably mRNA of the alpha-1 subunit or the alpha-3 subunit of NKA. Exemplary ribozymes contemplated herein include, without limitation, hammer head type ribozymes, ribozymes of the hairpin type, delta type ribozymes, etc. For teaching on ribozymes and design thereof, see, e.g., U.S. Pat. No. 5,354,855, U.S. Pat. No. 5,591,610, Pierce et al. 1998 (Nucleic Acids Res 26: 5093-5101), Lieber et al. 1995 (Mol Cell Biol 15: 540-551), and Benseler et al. 1993 (J Am Chem Soc 115: 8483-8484), incorporated herein by reference in their entirety.

In a yet further preferred embodiment, an agent capable of reducing the expression of the alpha-1 subunit and/or to the alpha-3 subunit of NKA is capable of causing RNA interference with the respective transcripts, preferably mRNAs.

“RNA interference” or “RNAi” is a term initially applied to a phenomenon observed in plants and worms where double-stranded RNA (dsRNA) blocks gene expression in a specific and post-transcriptional manner. Consequently, RNAi refers generally to the process of sequence-specific post-transcriptional gene silencing in animals mediated by short interfering nucleic acids (siNA), preferably by short interfering RNAs (siRNAs). RNAi provides a useful method of inhibiting gene expression in vitro or in vivo.

RNA interference agents may include any of short interfering nucleic acid (siNA), short interfering RNA (siRNA), double-stranded RNA (dsRNA), micro-RNA (miRNA), and short hairpin RNA (shRNA) molecules capable of mediating RNA interference (RNAi) against the expression of the alpha-1 subunit and/or to the alpha-3 subunit of NKA.

In the present context, the expression “dsRNA” relates to double stranded RNA capable of causing RNA interference. In accordance with the present invention, any suitable double-stranded RNA fragment capable of directing RNAi or RNA-mediated gene silencing of a target gene can be used. As used herein, a “double-stranded ribonucleic acid molecule (dsRNA)” refers to any RNA molecule, fragment or segment containing two strands forming an RNA duplex, notwithstanding the presence of single stranded overhangs of unpaired nucleotides. The double-stranded RNA comprises annealed complementary strands, one of which has a nucleotide sequence which corresponds to a target nucleotide sequence (i.e. to at least a portion of the mRNA transcript) of the target gene to be down-regulated. The other strand of the double-stranded RNA is complementary to this target nucleotide sequence.

The double-stranded RNA need only be sufficiently similar to the mRNA sequence of the target gene to be down-regulated that it has the ability to mediate RNAi. Thus, the invention has the advantage of being able to tolerate sequence variations that might be expected due to genetic mutation, strain polymorphism or evolutionary divergence. The number of tolerated nucleotide mismatches between the target sequence and a nucleotide sequence of the dsRNA sequence is no more than 1 in 5 basepairs, or 1 in 10 basepairs, or 1 in 20 basepairs, or 1 in 50 basepairs.

According to the invention, the “dsRNA” or “double stranded RNA”, whenever said expression relates to RNA that is capable of causing interference, may be formed form two separate (sense and antisense) RNA strands that are annealed together. Alternatively, the dsRNA may have a foldback stem-loop or hairpin structure wherein the two annealed strands of the dsRNA are covalently linked. In this embodiment, the sense and antisense strands of the dsRNA are formed from different regions of a single RNA sequence that is partially self-complementary.

As used herein, the term “RNAi molecule” is a generic term referring to double stranded RNA molecules including small interfering RNAs (siRNAs), hairpin RNAs (shRNAs), and other RNA molecules which can be cleaved in vivo to form siRNAs. RNAi molecules can comprise either long stretches of dsRNA identical or substantially identical to the target nucleic acid sequence or short stretches of dsRNA identical or substantially identical to only a region of the target nucleic acid sequence.

The subject RNAi molecules can be “small interfering RNAs” or “siRNAs.” siRNA molecules are usually synthesized as double stranded molecules in which each strand is around 19-30 nucleotides in length, and even more preferably 21-23 nucleotides in length. The siRNAs are understood to recruit nuclease complexes and guide the complexes to the target mRNA by pairing to the specific sequences. As a result, the target mRNA is degraded by the nucleases in the protein complex. In a particular embodiment, the siRNA molecules comprise a 3′ hydroxyl group. In certain embodiments, the siRNA molecules can be generated by processing of longer double-stranded RNAs, for example, in the presence of the enzyme dicer.

Alternatively, the RNAi molecule is in the form of a hairpin structure, named as hairpin RNA or shRNA. The hairpin RNAs can be synthesized exogenously or can be formed by transcribing from RNA polymerase III promoters in vivo. Preferably, such hairpin RNAs are engineered in cells or in an animal to ensure continuous and stable suppression of a desired gene. It is known in the art that siRNAs can be produced by processing a hairpin RNA in the cell.

The present RNAi molecules may include modifications to either the phosphate-sugar backbone or the nucleoside, e.g., to reduce susceptibility to cellular nucleases, improve bioavailability, improve formulation characteristics, and/or change other pharmacokinetic properties.

In some cases, at least one strand of the RNAi molecules has a 3′ overhang from about 1 to about 6 nucleotides in length, and for instance from 2 to 4 nucleotides in length. More preferably, the 3′ overhangs are 1-3 nucleotides in length. In certain embodiments, one strand has a 3′ overhang and the other strand is blunt-ended or also has an overhang. The length of the overhangs may be the same or different for each strand. In order to further enhance the stability of the RNAi molecules, the 3′ overhangs can be stabilized against degradation. In one embodiment, the RNA is stabilized by including purine nucleotides, such as adenosine or guanosine nucleotides. Alternatively, substitution of pyrimidine nucleotides by modified analogues, e.g., substitution of uridine nucleotide 3′ overhangs by 2′-deoxythymidine is tolerated and does not affect the efficiency of RNAi.

For further details on design of siRNA agents, see, e.g., Elbashir et al. 2001 (Nature 411: 494-501).

In a preferred embodiment, the invention relates to the use of an RNA sequence to prepare an RNAi molecule as defined herein, and preferably a siRNA molecule. Said siRNA molecule is characterized by one or more, and preferably by all of the following criteria:
having at least 50% sequence identity, preferably at least 70% sequence identity, more preferred at least 80% sequence identity, even more preferred at least 90% sequence identity with the target mRNA, e.g., mRNA for alpha-1 or alpha-3 subunit of NKA;
having a sequence which targets the exon area of the target gene;
showing a preference for targeting the 3′ end of the target gene rather than for targeting the 5′ end of the target gene.

In a further preferred embodiment, the siRNA molecule may be further characterized by one or more of the following criteria:
having a nucleic acid length of between 15 to 25 nucleotides and preferably of between 18 to 22 nucleotides, and preferably of 19 nucleotides;
having a GC content comprised between 30 and 50%
showing a TT(T) sequence at its 3′ end;
showing no secondary structure when adopting the duplex form;
having a Tm (melting temperature) of lower than 20° C.
having the nucleotides indicated in Table 2 in the sequence of the nucleotides, wherein h is a, c, t/u but not g, and wherein d is a, g, t/u but not c, and wherein w is a or t/u, but not g or c:

TABLE 2

| | | — | — | 1 | 2 | 3 | 4 | 5 | 6 | 7 | 8 | 9 | 10 | 11 | 12 | 13 | 14 | 15 | 16 | 17 | 18 | 19 | — | — | |
|---|---|---|---|---|---|---|---|---|---|---|---|---|---|---|---|---|---|---|---|---|---|---|---|---|---|
| mRNA | P′5 | A | A | | | A | | | | | | | U | | | h | | | | | | w | | | 3′-OH |
| si- | OH-3′ | T | T | | | U | | | | | | | A | | | d | | | | | | w | | | 5′-P |
| ASense | | | | | | | | | | | | | | | | | | | | | | | | | |
| si-Sense | P-5′ | | | | | A | | | | | | | U | | | h | | | | | | w | T | T | 3′-OH |

Production of any above nucleic acid reagents, including antisense reagents, ribozymes and RNAi molecules, can be carried out by chemical synthetic methods or by recombinant nucleic acid techniques, e.g., expressed from a vector in a cell, e.g., a viral vector, a eukaryotic expression vector, a gene therapy expression vector (i.e., in vivo), etc., or enzymatically synthesized, e.g., by in vitro transcription from a DNA template using a T7 or SP6 RNA polymerase. The nucleic acid molecules may be produced enzymatically or by partial/total organic synthesis. Any modified ribonucleotide can be introduced by in vitro enzymatic or organic synthesis.

Any above nucleic acid reagents, including antisense reagents, ribozymes and RNAi molecules, can be purified using a number of techniques known to those of skill in the art.

For example, gel electrophoresis can be used to purify nucleic acid reagents. Alternatively, non-denaturing methods, such as non-denaturing column chromatography, can be used to purify the molecules. In addition, chromatography (e.g., size exclusion chromatography), glycerol gradient centrifugation, affinity purification with antibody can be used to purify the molecules.

There are several well-known methods of introducing (ribo)nucteic acids (e.g., antisense, ribozymes or RNAi) into animal cells, any of which may be used in the present invention and which depend on the host. At the simplest, the nucleic acid can be directly injected into the target cell/target tissue. Other methods include fusion of the recipient cell with bacterial protoplasts containing the nucleic acid, the use of compositions like calcium chloride, rubidium chloride, lithium chloride, calcium phosphate, DEAE dextran, cationic lipids or liposomes or methods like receptor-mediated endocytosis, biolistic particle bombardment (“gene gun” method), infection with viral vectors, electroporation, and the like. Other techniques or methods which are suitable for delivering (ribo)nucleic acid molecules to target cells include the continuous delivery of an such molecule as defined herein from poly (lactic-Co-Glycolic Acid) polymeric microspheres or the direct injection of protected (stabilized) molecule(s) into micropumps delivering the product in the hole of surgical resection to the tumor cells still present at the site of surgery, e.g., in the hole of neurosurgical resection to the tumor cells still present in the brain parenchyma, as was detailed previously for the use of other anti-migratory compounds (Lefranc et al. 2003. Neurosurgery 52: 881-891). Convection-enhanced delivery, as detailed by Kawakami et al. 2004 (J Neurosurg 101: 1004-1011) of stabilized RNAi molecules as defined herein can also be used. Another possibility is the use of implantable drug-releasing biodegradable micropsheres, as those recently reviewed by Menei and Benoit 2003 (Acta Neurochir 88: 51-55). It shall be clear that also a combination of different above-mentioned delivery modes or methods may be used.

A preferred approach is to use either an Ommaya reservoir (micropumps) delivering the present RNAi molecule(s) versus encapsulated nucleic acids, e.g., RNAi molecules, in biodegradable microspheres, or both approaches at the same time.

The main obstacle to achieve in vivo gene silencing by nucleic acids, e.g., antisense, ribozyme or RNAi technologies, is delivery. To improve thermal stability, resistance to nuclease digestion and to enhance cellular uptake of such tools, various approaches are applicable and are known to a skilled person. They include, e.g.:
chemical modifications like locked nucleic acid (LNA), phosphonate substitution, phosphorothioate substitution, phosphorodithioate substitution, morpholino oligomers, 2′-fluoro substitution, 2′-O-methyl substitution, stabilized stealth™ RNAi (Invitrogen), etc.
encapsulation in various types of liposomes (immunoliposomes, PEGylated (immuno) liposomes), cationic lipids and polymers, nanoparticules or dendrimers, poly (lactic-Co-Glycolic Acid) polymeric microspheres, implantable drug-releasing biodegradable microspheres, etc.;
co-injection with protective agent like the nuclease inhibitor aurintricarboxylic acid.

Proliferative Disorders

The present invention concerns methods and agents useful for the treatment of proliferative disorders.

By “proliferative disease or disorder” is meant all neoplastic cell growth and proliferation, whether malignant or benign, including all transformed cells and tissues and all cancerous cells and tissues. Proliferative diseases or disorders include, but are not limited to, premalignant or precancerous lesions, abnormal cell growths, benign tumours, malignant tumours, and “cancer.”

Additional examples of proliferative diseases and/or disorders include, but are not limited to neoplasms, whether benign or malignant, located in the: prostate, colon, abdomen, bone, breast, digestive system, liver, pancreas, peritoneum, endocrine glands (adrenal, parathyroid, pituitary, testicles, ovary, thymus, thyroid), eye, head and neck, nervous (central and peripheral), lymphatic system, pelvic, skin, soft tissue, spleen, thoracic, and urogenital tract.

In a preferred embodiment, the proliferative disorder involves tumour.

As used herein, the terms “tumour” or “tumour tissue” refer to an abnormal mass of tissue that results from excessive cell division. A tumour or tumour tissue comprises “tumour cells” which are neoplastic cells with abnormal growth properties and no useful bodily function. Tumours, tumour tissue and tumour cells may be benign or malignant. A tumour or tumour tissue may also comprise “tumour-associated non-tumour cells”, e.g., vascular cells which form blood vessels to supply the tumour or tumour tissue. Non-tumour cells may be induced to replicate and develop by tumour cells, for example, the induction of angiogenesis in a tumour or tumour tissue.

In another preferred embodiment, the proliferative disorder involves malignancy or cancer.

As used herein, the term “malignancy” refers to a non-benign tumour or a cancer. As used herein, the term “cancer” connotes a type of proliferative disease which includes a malignancy characterized by deregulated or uncontrolled cell growth. Examples of cancer include, but are not limited to, carcinoma, lymphoma, blastoma, sarcoma, and leukemia or lymphoid malignancies. More particular examples of such cancers are noted below and include: squamous cell cancer (e.g., epithelial squamous cell cancer), lung cancer including small-cell lung cancer, non-small cell lung cancer, adenocarcinoma of the lung, squamous carcinoma of the lung and large cell carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial cancer or uterine carcinoma, salivary gland carcinoma, kidney or renal cancer, prostate cancer, vulval cancer, thyroid cancer, hepatic carcinoma, anal carcinoma, penile carcinoma, as well as head and neck cancer. The term “cancer” includes primary malignant cells or tumours (e.g., those whose cells have not migrated to sites in the subject's body other than the site of the original malignancy or tumour) and secondary malignant cells or tumours (e.g., those arising from metastasis, the migration of malignant cells or tumour cells to secondary sites that are different from the site of the original tumour).

Other examples of cancers or malignancies include, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumours, Breast Cancer, Cancer of the Renal Pelvis and Urethra, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Glioblastoma, Childhood Cerebellar Astrocytoma, Childhood Cerebral Astrocytoma, Childhood Extracranial Germ Cell Tumours, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumours, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumours, Exocrine Pancreatic Cancer, Extracranial Germ Cell Tumour, Extragonadal Germ Cell Tumour, Extrahepatic Bile Duct Cancer, Eye Cancer, Female Breast Cancer, Gaucher's Disease, Gallbladder Cancer, Gastric Cancer, Gastrointestinal Carcinoid Tumour, Gastrointestinal Tumours, Germ Cell Tumours, Gestational Trophoblastic Tumour, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumour, Ovarian Low Malignant Potential Tumour, Pancreatic Cancer, Paraproteinemias, Purpura, Parathyroid Cancer, Penile Cancer, Pheochromocytoma, Pituitary Tumour, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Urethra Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumours, T-Cell Lymphoma, Testicular Cancer, Thymoma, Thyroid Cancer, Transitional Cell Cancer of the Renal Pelvis and Urethra, Transitional Renal Pelvis and Urethra Cancer, Trophoblastic Tumours, Urethra and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumour, and any other proliferative disease, besides neoplasia, located in an organ system listed above.

In a further embodiment, the proliferative disorder is premalignant condition. Premalignant conditions are known or suspected of preceding progression to neoplasia or cancer, in particular, where non-neoplastic cell growth consisting of hyperplasia, metaplasia, or most particularly, dysplasia has occurred (for review of such abnormal growth conditions, see Robbins and Angell 1976 (Basic Pathology, 2d Ed., W. B. Saunders Co., Philadelphia, pp. 68-79).

“Hyperplasia” is a form of controlled cell proliferation, involving an increase in cell number in a tissue or organ, without significant alteration in structure or function. Hyperplastic disorders which can be treated by the method of the invention include, but are not limited to, angiofollicular mediastinal lymph node hyperplasia, angiolymphoid hyperplasia with eosinophilia, atypical melanocytic hyperplasia, basal cell hyperplasia, benign giant lymph node hyperplasia, cementum hyperplasia, congenital adrenal hyperplasia, congenital sebaceous hyperplasia, cystic hyperplasia, cystic hyperplasia of the breast, denture hyperplasia, ductal hyperplasia, endometrial hyperplasia, fibromuscular hyperplasia, focal epithelial hyperplasia, gingival hyperplasia, inflammatory fibrous hyperplasia, inflammatory papillary hyperplasia, intravascular papillary endothelial hyperplasia, nodular hyperplasia of prostate, nodular regenerative hyperplasia, pseudoepitheliomatous hyperplasia, senile sebaceous hyperplasia, and verrucous hyperplasia.

“Metaplasia” is a form of controlled cell growth in which one type of adult or fully differentiated cell substitutes for another type of adult cell. Metaplastic disorders which can be treated by the method of the invention include, but are not limited to, agnogenic myeloid metaplasia, apocrine metaplasia, atypical metaplasia, autoparenchymatous metaplasia, connective tissue metaplasia, epithelial metaplasia, intestinal metaplasia, metaplastic anemia, metaplastic ossification, metaplastic polyps, myeloid metaplasia, primary myeloid metaplasia, secondary myeloid metaplasia, squamous metaplasia, squamous metaplasia of amnion, and symptomatic myeloid metaplasia.

“Dysplasia” is frequently a forerunner of cancer, and is found mainly in the epithelia; it is the most disorderly form of non-neoplastic cell growth, involving a loss in individual cell uniformity and in the architectural orientation of cells. Dysplastic cells often have abnormally large, deeply stained nuclei, and exhibit pleomorphism. Dysplasia characteristically occurs where there exists chronic irritation or inflammation. Dysplastic disorders which can be treated by the method of the invention include, but are not limited to, anhidrotic ectodermal dysplasia, anterofacial dysplasia, asphyxiating thoracic dysplasia, atriodigital dysplasia, bronchopulmonary dysplasia, cerebral dysplasia, cervical dysplasia, chondroectodermal dysplasia, deidocranial dysplasia, congenital ectodermal dysplasia, craniodiaphysial dysplasia, craniocarpotarsal dysplasia, craniometaphysial dysplasia, dentin dysplasia, diaphysial dysplasia, ectodermal dysplasia, enamel dysplasia, encephalo-ophthalmic dysplasia, dysplasia epiphysialis hemimelia, dysplasia epiphysialis multiplex, dysplasia epiphysialis punctata, epithelial dysplasia, faciodigitogenital dysplasia, familial fibrous dysplasia of jaws, familial white folded dysplasia, fibromuscular dysplasia, fibrous dysplasia of bone, florid osseous dysplasia, hereditary renal-retinal dysplasia, hidrotic ectodermal dysplasia, hypohidrotic ectodermal dysplasia, lymphopenic thymic dysplasia, mammary dysplasia, mandibulofacial dysplasia, metaphysial dysplasia, Mondini dysplasia, monostotic fibrous dysplasia, mucoepithelial dysplasia, multiple epiphysial dysplasia, oculoauriculovertebral dysplasia, oculodentodigital dysplasia, oculovertebral dysplasia, odontogenic dysplasia, ophthalmomandibulomelic dysplasia, periapical cemental dysplasia, polyostotic fibrous dysplasia, pseudoachondroplastic spondyloepiphysial dysplasia, retinal dysplasia, septo-optic dysplasia, spondyloepiphysial dysplasia, and ventriculoradial dysplasia.

Additional pre-neoplastic disorders include, but are not limited to, benign dysproliferative disorders (e.g., benign tumours, fibrocystic conditions, tissue hypertrophy, intestinal polyps, colon polyps, and oesophageal dysplasia), leukoplakia, keratoses, Bowen's disease, Farmer's Skin, solar cheilitis, and solar keratosis.

In preferred embodiments, the proliferative disorder is chosen from glioma, preferably glioblastoma; prostate cancer; non-small-cell lung cancer (NSCLC); or colon cancer. The inventors realised that the above cancer types can particularly benefit from the methods and agents of the invention.

As used herein, the term “glioma” refers to its art-recognised connotation. By virtue of further illustration and not limitation, the term “glioma” refers to a tumour originating in the neuroglia of the brain or spinal cord. Gliomas can be derived from glial cell types, such as, e.g., astrocytes and oligodendrocytes, thus gliomas include astrocytomas and oligodendrogliomas, as well as anaplastic gliomas, glioblastomas, and ependymonas. Astrocytomas and ependymomas can occur in all areas of the brain and spinal cord in both children and adults. Oligodendrogliomas typically occur in the cerebral hemispheres of adults. Malignant astrocytic gliomas are associated with the worst prognoses because of their ability to infiltrate diffusely into the normal brain parenchyma and include World Health Organization (WHO) grades II, III and grade IV tumors.

As used herein, the term “glioblastoma” refers to its art-recognised connotation. By virtue of further illustration and not limitation, glioblastoma may also be known as “glioblastoma multiforme” (GBM) or as “grade 4 astrocytoma” and represents perhaps the most common and aggressive type of malignant primary brain tumour.

As used herein, the term “prostate cancer” (CaP) refers to its art-recognised connotation. By virtue of illustration and not limitation, the term “prostate cancer” refers to both the appearance of a palpable tumour of the prostate, and also to microscopically detectable neoplastic or transformed cells in the prostate gland. In the latter case, the said cytologically-detectable prostate cancer may be asymptomatic, in that neither the patient nor the medical practitioner detects the presence of the cancer cells. Cancer cells are generally found in the prostates of men who live into their seventies or eighties, however not all of these men develop prostate cancer. In the event that prostate cancer metastasises to additional sites distal to the prostate, the condition is described as metastatic cancer (MC), to distinguish this condition from organ-confined prostate cancer. CaP fatality typically results from metastatic dissemination of prostatic adenocarcinoma cells to distant sites, usually in the axial skeleton.

The term “non-small-cell lung cancer” (NSCLC) refers to its art-recognised connotation. By means of exemplification and not limitation, the term encompasses any of subtypes thereof, i.e., adenocarcinoma of the lung, squamous cell carcinoma of the lung and large cell carcinoma of the lung.

The term “colon cancer” refers to its art-recognised connotation. By means of illustration and not limitation, the term “colon cancer” refers to cancers arising in the large intestine (including both the colon and rectum) of any histologic type, including but not limited to malignant epithelial tumours. As used herein the term colon cancer thus encompasses colorectal cancer. Malignant epithelial tumours of the large intestine may be divided into five major histologic types: adenocarcinoma, mucinous adenocarcinoma (also termed colloid adenocarcinoma), signet ring adenocarcinoma, scirrhous tumours and carcinoma simplex. Colon cancer is staged using any of several classification systems known in the art. The Dukes system is one of the most often employed staging systems. See Dukes and Bussey 1958 (Br J Cancer 12: 309).

In a further preferred embodiment, the proliferative disorder is one that overexpresses the alpha-1 subunit and/or the alpha-3 subunit of the NKA.

A proliferative disorder, e.g., cancer or any of the above, which “overexpresses” the alpha-1 subunit and/or the alpha-3 subunit of the NKA is one which, per gross mass and/or at the level of individual cells, produces significantly higher levels of the said subunits(s) compared to a healthy, e.g., non-cancerous, cells of the same tissue type. Overexpression of the said subunit(s) may be determined diagnostically by evaluating the levels of the subunit(s) polypeptide(s) and/or nucleic acid(s) encoding such in a sample from a patient, e.g., in a tumour biopsy. Techniques generally employable in such determination are well-known in the art and include, without limitation, IHC, FISH, southern blotting, PCR, Western blotting, ELISA, RIA, immunoprecipitation, etc. The term encompasses any extent of overexpression, such as, by way of example, overexpression by at least about 10%, e.g., by at least about 20%, preferably by at least about 30%, e.g., by at least about 40%, more preferably by at least about 50%, e.g., by at least about 60%, even more preferably by at least about 70%, e.g., by at least about 80%, yet more preferably by at least about 90%, e.g., by at least about 100%, or even higher, e.g., by at least about 150%, at by least about 200%, by at least about 250%, by at least about 300%, by at least about 400% or even by at least about 500%, e.g., as measured in gross mass and/or at the level of individual cells.

In a further preferred embodiment, the proliferative disorder which overexpresses the alpha-1 subunit and/or the alpha-3 subunit of the NKA is chosen from glioma, preferably glioblastoma; prostate cancer; non-small-cell lung cancer (NSCLC); or colon cancer. The inventors realised that these cancer types often overexpress the alpha-1 subunit and/or the alpha-3 subunit of the NKA.

In a further preferred embodiment, the proliferative disorder which overexpresses the alpha-1 subunit of the NKA is non-small-cell lung cancer. The inventors realised that this cancer type particularly often overexpresses the alpha-1 subunit of the NKA.

Treatment

The present invention also regards treating proliferative disorders in a subject needing such therapy, comprising administering a therapeutically effective amount of one or more above agent(s) of the invention.

Except when noted, “subject” or “patient” are used interchangeably and refer to animals, preferably vertebrates, more preferably mammals, and specifically includes human patients and non-human mammals. “Mammalian” subjects include, but are not limited to, humans, domestic animals, farm animals, zoo animals, sport animals, pet and experimental animals such as dogs, cats, guinea pigs, rabbits, rats, mice, horses, cattle, cows; primates such as apes, monkeys, orang-utans, and chimpanzees; canids such as dogs and wolves; felids such as cats, lions, and tigers; equids such as horses, donkeys, and zebras; food animals such as cows, pigs, and sheep; ungulates such as deer and giraffes; rodents such as mice, rats, hamsters and guinea pigs; and so on. Accordingly, “subject” or “patient” as used herein means any mammalian patient or subject to which the compositions of the invention can be administered.

Preferred patients are human subjects.

As used herein, the terms “treat” or “treatment” refer to both therapeutic treatment and prophylactic or preventative measures, wherein the object is to prevent or slow down (lessen) an undesired physiological change or disorder, such as the development or spread of proliferative disease, e.g., cancer. Beneficial or desired clinical results include, but are not limited to, alleviation of symptoms, diminishment of extent of disease, stabilised (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment.

As used herein, a phrase such as “a subject in need of treatment” includes subjects, such as mammalian subjects, that would benefit from treatment of a given condition, preferably a proliferative disease, such as, e.g., cancer, e.g., as above. Such subjects will typically include, without limitation, those that have been diagnosed with the condition, preferably a proliferative disease, e.g., cancer, those prone to have or develop the said condition and/or those in whom the condition is to be prevented.

The term “therapeutically effective amount” refers to an amount of a therapeutic substance or composition effective to treat a disease or disorder in a subject, i.e., to obtain a desired local or systemic effect and performance. By means of example and not limitation, in the case of proliferative disease, e.g., cancer, therapeutically effective amount of a drug may reduce the number of cancer cells; reduce the tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; enhance efficacy of another cancer therapy; and/or relieve to some extent one or more of the symptoms associated with the cancer. To the extent the drug may prevent growth and/or kill existing cancer cells, it may be cytostatic and/or cytotoxic. For cancer therapy, efficacy can, for example, be measured by assessing the time to disease progression (TTP) and/or determining the response rate (RR).

The agent(s) of the invention may be used alone or in combination with any of the cancer therapies selected from the group comprising chemotherapy, radiation therapy, immunotherapy, and/or gene therapy. As used herein the term “cancer therapy” is meant to encompass radiation therapy, chemotherapy, immunotherapy, gene-based therapy, surgery, as well as combinations thereof.

In another preferred embodiment the agents of the invention may be used alone or in combination with one or more active compounds that are suitable in the treatment of cancer, preferably glioma, preferably glioblastoma; prostate cancer; NSCLC; or colon cancer. The term “active compound” refers to a compound other than the agents of the invention which is used to treat cancer. The active compounds may preferably be selected from the group comprising radiation therapeutics, chemotherapeutics including but not limited to temozolomide, vincristine, vinorelbine, procarbazine, carmustine, lomustine, taxol, taxotere, tamoxifen, retinoic acid, 5-fluorouracil, cyclophosphamide and thalidomide, immunotherapeutics such as but not limited to activated T cells and pulsed dendritic cells, and/or gene-based therapeutic approached involving gene transfer of CD3, CD7 and CD45 in glioma cells, concomitantly with the delivery of the agents of the invention.

The agent(s) of the invention can thus be administered alone or in combination with one or more active compounds. The latter can be administered before, after or simultaneously with the administration of the said agent(s).

Pharmaceutical Preparations

A further object of the invention are pharmaceutical preparations which comprise a therapeutically effective amount an agent or agent(s) of the invention as defined herein, or pharmaceutically acceptable salts thereof, and a pharmaceutically acceptable carrier, i.e., one or more pharmaceutically acceptable carrier substances and/or additives, e.g., buffers, carriers, excipients, stabilisers, etc.

The term “pharmaceutically acceptable” as used herein is consistent with the art and means compatible with the other ingredients of a pharmaceutical composition and not deleterious to the recipient thereof.

The term “pharmaceutically acceptable salts” as used herein means an inorganic acid addition salt such as hydrochloride, sulfate, and phosphate, or an organic acid addition salt such as acetate, maleate, fumarate, tartrate, and citrate. Examples of pharmaceutically acceptable metal salts are alkali metal salts such as sodium salt and potassium salt, alkaline earth metal salts such as magnesium salt and calcium salt, aluminum salt, and zinc salt. Examples of pharmaceutically acceptable ammonium salts are ammonium salt and tetramethylammonium salt. Examples of pharmaceutically acceptable organic amine addition salts are salts with morpholine and piperidine. Examples of pharmaceutically acceptable amino acid addition salts are salts with lysine, glycine, and phenylalanine.

The pharmaceutical composition according to the invention may further comprise at least one active compound, as defined above.

The pharmaceutical composition according to the invention can be administered orally, for example in the form of pills, tablets, lacquered tablets, sugar-coated tablets, granules, hard and soft gelatin capsules, aqueous, alcoholic or oily solutions, syrups, emulsions or suspensions, or rectally, for example in the form of suppositories. Administration can also be carried out parenterally, for example subcutaneously, intramuscularly or intravenously in the form of solutions for injection or infusion. Other suitable administration forms are, for example, percutaneous or topical administration, for example in the form of ointments, tinctures, sprays or transdermal therapeutic systems, or the inhalative administration in the form of nasal sprays or aerosol mixtures, or, for example, microcapsules, implants or rods.

The preparation of the pharmaceutical compositions can be carried out in a manner known per se. To this end, the nucleic acid and/or the active compound, together with one or more solid or liquid pharmaceutical carrier substances and/or additives (or auxiliary substances) and, if desired, in combination with other pharmaceutically active compounds having therapeutic or prophylactic action, are brought into a suitable administration form or dosage form which can then be used as a pharmaceutical in human medicine. For the production of pills, tablets, sugar-coated tablets and hard gelatin capsules it is possible to use, for example, lactose, starch, for example maize starch, or starch derivatives, talc, stearic acid or its salts, etc. Carriers for soft gelatin capsules and suppositories are, for example, fats, waxes, semisolid and liquid polyols, natural or hardened oils, etc. Suitable carriers for the preparation of solutions, for example of solutions for injection, or of emulsions or syrups are, for example, water, physiological sodium chloride solution, alcohols such as ethanol, glycerol, polyols, sucrose, invert sugar, glucose, mannitol, vegetable oils, etc. It is also possible to lyophilize the nucleic acid and/or the active compound and to use the resulting lyophilisates, for example, for preparing preparations for injection or infusion. Suitable carriers for microcapsules, implants or rods are, for example, copolymers of glycolic acid and lactic acid.

The pharmaceutical preparations can also contain additives, for example fillers, disintegrants, binders, lubricants, wetting agents, stabilizers, emulsifiers, dispersants, preservatives, sweeteners, colorants, flavorings, aromatizers, thickeners, diluents, buffer substances, solvents, solubilizers, agents for achieving a depot effect, salts for altering the osmotic pressure, coating agents or antioxidants.

Preferably, the present composition is administered in a GLP/GMP solvent, containing or not cyclobetadextrine and/or similar compounds.

The dosage or amount of agents of the invention used, optionally in combination with one or more active compounds to be administered, depends on the individual case and is, as is customary, to be adapted to the individual circumstances to achieve an optimum effect. Thus, it depends on the nature and the severity of the disorder to be treated, and also on the sex, age, weight and individual responsiveness of the human or animal to be treated, on the efficacy and duration of action of the compounds used, on whether the therapy is acute or chronic or prophylactic, or on whether other active compounds are administered in addition to the agent(s) of the invention.

Without limitation, depending on the type and severity of the disease, a typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. A preferred dosage of the agent may be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks.

Where applicable, e.g., where the agent is a polypeptide, peptide, antibody, antisense agent, ribozyme or siRNA agent, the invention also contemplates administration thereof by gene therapy, according to effective techniques known in the art.

By way of example, the agents of the invention may be delivered at the site of the tumor, e.g., the primary tumor and/or metastases. A manner of achieving localized delivery is the use of the Ommaya reservoir as described elsewhere.

In another embodiment, the invention provides a kit comprising a pharmaceutical composition according to the invention, and an active compound as defined herein, for simultaneous, separate or sequential administration to a subject in need thereof.

Screening Assays

In an aspect, the invention provides assays to select, from a group of test agents, a candidate agent potentially useful as a therapeutic in the treatment of a proliferative disorder, said assay comprising determining whether the tested agent (a) can reduce the expression of the alpha-1 subunit of Na⁺,K⁺-ATPase or (b) can bind to the alpha-1 subunit of Na⁺,K⁺-ATPase, and/or whether the tested agent (c) can reduce the expression of the alpha-3 subunit of Na⁺,K⁺-ATPase or (d) can bind to the alpha-3 subunit of Na⁺,K⁺-ATPase.

Preferred types of test agents in the screening assays are agents as described in the previous sections, including antisense agents, e.g., antisense oligonucleotides, ribozymes, agents potentially capable of causing RNA interference; polypeptides or proteins; antibodies; peptides, peptidomimetics, aptamers, chemical substances (preferably an organic molecules, more preferably a small organic molecules), lipids, carbohydrates, nucleic acids, etc. Some of said test agent types, e.g., chemical compounds, peptides, carbohydrates, etc., can be obtained from synthetic, combinatorial or natural product libraries. Other test agents may be designed with the knowledge of their target.

In an embodiment, the assays (drug screening assays or bioassays) include a step of assessing the test agent for its ability to bind to the alpha-1 subunit and/or to the alpha-3 subunit of NKA or to a variant thereof, preferably a functional and/or immunologicaly active variant thereof, or to a fragment thereof, preferably a functional and/or immunolgically active fragment thereof, or to a functional and/or immunologically active derivative thereof.

The term “variant” of alpha-1 subunit or of alpha-3 subunit of NKA, as used herein, refers to polypeptides the amino acid sequence of which is substantially identical (i.e., largely but not wholly identical) to a native sequence of, respectively, alpha-1 or alpha-3 subunit of NKA. “Substantially identical” refers to at least 85% identical, e.g., preferably at least 90% identical, e.g., at least 91% identical, 92% identical, more preferably at least 93% identical, e.g., 94% identical, even more preferably at least 95% identical, e.g., at least 96% identical, yet more preferably at least 97% identical, e.g., at least 98% identical, and most preferably at least 99% identical.

Sequence identity between two polypeptides can be determined by optimally aligning (optimal alignment of two protein sequences is the alignment that maximises the sum of pair-scores less any penalty for introduced gaps; and may be preferably conducted by computerised implementations of algorithms, such as “Gap”, using the algorithm of Needleman and Wunsch 1970 (J Mol Biol 48: 443-453), or “Bestfit”, using the algorithm of Smith and Waterman 1981 (J Mol Biol 147: 195-197), as available in, e.g., the GCG™ v. 11.1.2 package from Accelrys) the amino acid sequences of the polypeptides and scoring, on one hand, the number of positions in the alignment at which the polypeptides contain the same amino acid residue and, on the other hand, the number of positions in the alignment at which the two polypeptides differ in their sequence. The two polypeptides differ in their sequence at a given position in the alignment when the polypeptides contain different amino acid residues at that position (amino acid substitution), or when one of the polypeptides contains an amino acid residue at that position while the other one does not or vice versa (amino acid insertion or deletion). Sequence identity is calculated as the proportion (percentage) of positions in the alignment at which the polypeptides contain the same amino acid residue versus the total number of positions in the alignment. Further suitable algorithms for performing sequence alignments and determination of sequence identity include those based on the Basic Local Alignment Search Tool (BLAST) originally described by Altschul et al. 1990 (J Mol Biol 215: 403-10), such as the “Blast 2 sequences” algorithm described by Tatusova and Madden 1999 (FEMS Microbiol Lett 174: 247-250).

At least some of the differences between the amino acid sequences of a variant and of the naturally occurring alpha-1 or alpha-3 subunit with which the variant is substantially identical, can involve amino acid substitutions. Preferably, at least 85%, e.g., at least 90%, more preferably at least 95%, e.g., 100% of the said differences can be amino acid substitutions, preferably conservative amino acid substitutions. The term “conservative substitution” as used herein denotes that one amino acid residue has been replaced by another, biologically similar amino acid residue. Non-limiting examples of conservative substitutions include the substitution of one hydrophobic amino acid residue, such as isoleucine, valine, leucine or methionine for another, or the substitution of one polar residue for another, such as between arginine and lysine, between glutamic and aspartic acids or between glutamine and asparagine, and the like.

The “variant” of alpha-1 subunit or of alpha-3 subunit of NKA, as used herein, also specifically includes polypeptides having a certain degree of similarity to, respectively, alpha-1 or alpha-3 subunit of NKA. Preferably, such variants can be at least 90% similar, e.g., preferably at least 91% similar, e.g., at least 92% similar, 93% similar, more preferably at least 94% similar, e.g., 95% similar, even more preferably at least 96% similar, e.g., at least 97% similar, yet more preferably at least 98% similar, e.g., at least 99% similar.

Sequence similarity between two polypeptides can be determined by optimally aligning (see above) the amino acid sequences of the polypeptides and scoring, on one hand, the number of positions in the alignment at which the polypeptides contain the same or similar (i.e., conservatively substituted) amino acid residue and, on the other hand, the number of positions in the alignment at which the two polypeptides otherwise differ in their sequence. The two polypeptides otherwise differ in their sequence at a given position in the alignment when the polypeptides contain non-conservative amino acid residues at that position, or when one of the polypeptides contains an amino acid residue at that position while the other one does not or vice versa (amino acid insertion or deletion). Sequence similarity is calculated as the proportion (percentage) of positions in the alignment at which the polypeptides contain the same or similar amino acid residue versus the total number of positions in the alignment.

The term “functional variant” of alpha-1 subunit or of alpha-3 subunit of NKA as used herein refers to a variant as defined above which at least partly retains its functionality within Na⁺,K⁺-ATPase. For example, Na⁺,K⁺-ATPase containing one or two of such variant alpha-1 subunit or variant alpha-3 subunit would retain at least 20%, e.g., at least 30% or at least 40%, preferably at least 50%, e.g., at least 60%, more preferably at least 70%, even more preferably at least 80%, and most preferably at least 90%, e.g., at least 95% of its enzymatic activity, as measured by standard assays in the art.

The term “fragment” of alpha-1 subunit or of alpha-3 subunit of NKA, as used herein, refers to a polypeptide that has an N-terminal and/or C-terminal deletion of one or more amino acid residues as compared to the NKA alpha-1 subunit or NKA alpha-3 subunit, or a variant (preferably a functional variant) of any thereof, but where the remaining primary sequence of the fragment is identical to the corresponding positions in the amino acid sequence of the NKA alpha-1 subunit or NKA alpha-3 subunit, or a variant (preferably a functional variant) thereof, respectively.

For example, a fragment of alpha-1 subunit or of alpha-3 subunit, or of a (preferably functional) variant thereof, may include a sequence of ≧5 consecutive amino acids, preferably ≧10 consecutive amino acids, more preferably ≧20 consecutive amino acids, even more preferably ≧30 consecutive amino acids, e.g., ≧40 consecutive amino acids, and most preferably ≧50 consecutive amino acids, e.g., ≧60, ≧70, ≧80, ≧90, ≧100, ≧200 or ≧500 consecutive amino acids of, respectively, the said alpha-1 subunit or alpha-3 subunit, or of a (preferably functional) variant thereof.

A fragment of alpha-1 subunit or of alpha-3 subunit, or of a (preferably functional) variant thereof, can also represent at least 80%, e.g., at least 85%, preferably at least 90%, more preferably at least 95% or even 99% of the amino acid sequence of, respectively, the said alpha-1 subunit or alpha-3 subunit, or of a (preferably functional) variant thereof.

The term “functional fragment” of alpha-1 subunit or of alpha-3 subunit of NKA, as used herein, refers to a fragment as defined above which at least partly retains its functionality within Na⁺,K⁺-ATPase. For example, Na⁺,K⁺-ATPase containing one or two of such alpha-1 subunit fragments or alpha-3 subunit fragments would retain at least 20%, e.g., at least 30% or at least 40%, preferably at least 50%, e.g., at least 60%, more preferably at least 70%, even more preferably at least 80%, and most preferably at least 90%, e.g., at least 95% of its enzymatic activity, as measured by standard assays in the art.

Typically, an embodiment includes (a) combining (1) the alpha-1 subunit or alpha-3 subunit of NKA, or a variant, fragment or derivative thereof (preferably functional and/or immunologically active) and (2) a test agent, e.g., under conditions which allow for binding of the polypeptide (1) and test agent (2) to form a complex, and detecting the formation of a complex, in which the ability of the test agent (2) to interact with polypeptide (1) is indicated by the presence of the test agent in the complex. Formation of said complexes can be quantified, for example, using standard immunoassays. The embodiment may further comprise isolation and/or identification of the said test agent.

The alpha-1 subunit or alpha-3 subunit of NKA, variants, fragments or derivatives thereof (preferably functional and/or immunologically active) used in such assays may be free in solution, affixed to a solid support, born on a cell surface, or located intracellularly. The method may use eukaryotic or prokaryotic host cells which natively express alpha-1 or alpha-3 subunits, or which are transiently or stably transformed with recombinant nucleic acids expressing the said subunits or variants, fragments or derivative thereof.

The invention also contemplates competitive screening assays in which neutralizing antibodies or compounds (e.g., ouabain, digoxin) capable of binding the alpha-1 subunit or the alpha-3 subunit of NKA, variants, fragments or derivatives thereof (preferably functional to and/or immunologically active) compete with a test agent for binding the said subunits. In particular, the present invention pertains to a competitive screening assay comprising: (a) competing an antibody or compound specific for the alpha-1 subunit or the alpha-3 subunit of NKA, variant, fragment or derivative thereof (preferably functional and/or immunologically active) with a test agent for binding to the said polypeptides, and (b) determining the amount of competition of said antibody compared to said test agent. The above screening assays may further comprise determining the specificity of a test agent for binding to the alpha-1 or alpha-3 subunit, by comparing the strength of such binding to the strength of binding of the said agent to other cellular proteins, esp. to other alpha subunits of NKA.

The above screening assays may further comprise step of assessing whether a test agent, preferably a test agent that binds the alpha-1 and/or the alpha-3 subunit of NKA also alters, e.g., inhibits or activates, the biological activity, e.g., enzymatic activity of said NKA. Typically, said method may comprise contacting the test agent with a cell, tissue, organ or non-human model organism expressing the alpha-1 subunit or the alpha-3 subunit of NKA or functional variants, fragments or derivatives thereof and having NKA activity, and assessing alteration in biological activity of the NKA. Suitable assessment methods are described, e.g., in examples 3, 4 and 6.

In an embodiment, the assays (drug screening assays or bioassays) include a step of assessing the test agent for its ability to reduce the expression of the alpha-1 subunit and/or of the alpha-3 subunit of NKA. In an embodiment, said assay comprises: (a) providing a cell expressing the alpha-1 subunit or the alpha-3 subunit of NKA, or optionally a variant, derivative or fragment thereof, (b) introducing to said cell a test agent, and (c) determining the expression of the alpha-1 subunit or the alpha-3 subunit of NKA, or optionally a variant, derivative or fragment thereof, thereby identifying whether the test agent modulates the said expression. Expression can be quantified at various levels as described above. The embodiment may further comprise isolation and/or identification of the said test agent. The expression of the alpha-1 subunit or the alpha-3 subunit in the cell may be intrinsic to the cell or may be facilitated recombinantly, e.g., by transforming the said cell transiently or stably with a nucleic acid, e.g., cDNA, encoding the alpha-1 subunit or the alpha-3 subunit or a suitable variant, fragment or derivative thereof.

The above screening assays may further comprise step of assessing whether a test agent, preferably a test agent that reduces the expression of alpha-1 and/or the alpha-3 subunit of NKA also alters the biological activity, e.g., enzymatic activity of said NKA. Typically, said method may comprise contacting the test agent with a cell, tissue, organ or non-human model organism expressing the alpha-1 subunit or the alpha-3 subunit of NKA or functional variants, fragments or derivatives thereof and having NKA activity, and assessing alteration in biological activity of the NKA. Suitable assessment methods are described, e.g., in examples 3, 4 and 6.

In embodiments, the assays are to select, from a group of test agents, a candidate agent potentially useful as a therapeutic in the treatment of a proliferative disorder chosen from glioma, preferably glioblastoma; prostate cancer; non-small-cell lung cancer (NSCLC); or colon cancer, as defined above.

In an embodiment, the assays are to select, from a group of test agents, a candidate agent potentially useful as a therapeutic in the treatment of non-small-cell lung cancer (NSCLC), as defined above.

In a further embodiment, the assays are to select, from a group of test agents, a candidate agent potentially useful as a therapeutic in the treatment of a proliferative disorder that overexpresses the alpha-1 subunit and/or the alpha-3 subunit of the NKA.

In further preferred embodiments, the assays are to select, from a group of test agents, a candidate agent potentially useful as a therapeutic in the treatment of the proliferative disorder which overexpresses the alpha-1 subunit and/or the alpha-3 subunit of the NKA and is chosen from glioma, preferably glioblastoma; prostate cancer; non-small-cell lung cancer (NSCLC); or colon cancer.

In a further preferred embodiment, the assays are to select, from a group of test agents, a candidate agent potentially useful as a therapeutic in the treatment of the proliferative disorder which overexpresses the alpha-1 subunit of NKA and is non-small-cell lung cancer.

In addition, the invention also relates to the agents identifiable by any of the herein described screening methods. Also, the present invention contemplates a method for the production of a composition comprising the steps of admixing an agent identifiable by the assays as described herein with a pharmaceutically acceptable carrier. It will be clear that the present invention contemplates a composition comprising an agent identifiable by any of the herein described methods. Moreover, the present invention contemplates the use of an agent to identifiable by any of the herein described methods as medicament. Such agents are particularly suited for the treatment of proliferative disorders as defined herein, particularly cancers, e.g., cancers overexpressing the alpha-1 or the alpha-3 subunit of NKA, e.g., glioma, glioblastoma, prostate cancer, NSCLC or colon cancer.

The invention is further illustrated with examples that are not to be considered limiting.

### Examples

### Example 1

### Expression of Alpha-1 NKA Subunit is Increased in NSCLC

The cell lines under study obtained from the American Type Culture Collection (Manassas, Va.) included: two human NSCLC models, i.e. A549 (ATCC code CCL-185) and A427 (ATCC code HTB-53); two normal human lung fibroblast cell lines, i.e. WI-38 (ATCC code CCL-75) and ccd25-Lu (ATCC code CCL-215); mouse melanoma cell line B16F10 (ATCC code CRL-6475); and rat glioma C6 cell line (ATCC code CCL-107). The CAL-12T cells were obtained from the DSMZ Animal Cell Line Database (code ACC-443; Brunswick, Germany). The NCI-H727 cells were obtained from the European Collection of Cell Cultures (code ECACC 94060303; Sigma-Aldrich, Bornem, Belgium). The mouse MXT mammary cancer cell line was established in our laboratory (Kiss et al. Cancer Res 49:2945-2951, 1989).

Eighty four archival (formalin-fixed and paraffin-embedded) lung tissues were provided by Dr. I. Salmon (Department of Pathology, Erasme University Hospital, Brussels, Belgium) and are part of a series of cases for which all clinicopathological data was described (Mathieu et al. 2005. Mod Pathol 18: 1264-1271). 25 normal tissues were obtained in the margins of the surgically resected NSCLC. The remaining 59 samples included 30 adenocarcinomas of NSCLC origin (NSCLC-ADC) and 29 squamous cell carcinomas of NSCLC origin (NSCLC-SCC). The 60 NSCLCs under study are from 59 patients who underwent the surgical resection of their NSCLC at the Erasmus University Hospital between 1995 and 2001. The clinical data available are summarized in Table 1. The tumors were classified according to the TNM classification (UICC 2002; 23) and staging was performed as follows: stage I (T1-2 N0 M0), stage II (T1-2 N1 M0 or T3 N0 M0), stage III (T1-2 N2-3 M0, T3 N1-3 M0 or T4 N0-3 M0) and stage IV (any T and N and M1).

TABLE 3
Clinicopathological data for 59 patients with primary squamous
cell carcinomas or adenocarcinomas

| | |
|---|---|
| Age (years), median (range) | 64 (43-78) |
| Gender, female/male | 14/45 |
| Smoking habits^{a} | |
| Smoker | 51 (94%) |
| Nonsmoker | 3 (6%) |
| Neoadjuvant therapy (4/59) | |
| Chemotherapy | 4 |
| Radiotherapy | 0 |
| Radiochemotherapy | 0 |
| Surgical treatment | |
| Segmentectomy | 4 (7%) |
| Lobectomy | 46 (78%) |
| Pneumonectomy | 9 (15%) |
| Histology | |
| Squamous cell carcinoma | 30 (51%) |
| Well differentiated | 4 |
| Moderately differentiated | 14 |
| Poorly differentiated | 11 |
| Adenocarcinoma | 29 (49%) |
| cinar | 22 |
| Papillary | 1 |
| Solid | 4 |
| Mixed | 3 |

^{a}smoking habits known for 54 cases

The 84 human tissue samples are from a retrospective analysis of formalin-fixed paraffin-embedded archival material. In the case of the six cell lines under study (the two normal fibroblast cell lines and the four NSCLC cell lines), we obtained cell pellets by centrifugating 10 million cells from each of the six cell lines for 10 minutes at 800×g, as detailed elsewhere (Sunaga et al. 2004. Cancer Res 64: 4277-4285). These pellets were then fixed for 20 min in buffered formalin (4%), dehydrated, and embedded in paraffin wax. Three pellets were available for each of the six cell lines.

Before starting immunohistochemistry dewaxed tissue sections were submitted to antigen retrieval for 2×5 min at 600 W in Dakocytomation buffer (pH6.1) (DAKO, Glostrup, Denmark). The sections were then incubated with a solution of 0.4% hydrogen peroxide in methanol for 30 min to block endogenous peroxidase activity, rinsed in phosphate-buffered saline (PBS; 0.04 M Na₂HPO₄, 0.01 M KH₂PO₄ and 0.12 M NaCl, pH 7.4) The sections were then sequentially exposed at room temperature i) to the specific primary antibodies for one hour (see below); ii) to the secondary antibody (Ultrasense Biotynilated Goat anti-polyvalent RTU, ImmunoLogic, Duiven, The Nederlands) iii) to the Ultrasense Streptavidine Peroxydase RTU (ImmunoLogic, Duiven, The Nederlands) for 10 min and iv) to the of avidin-biotin-peroxidase complex (ABC kit, Glostrup, Denmark). The antigen-dependent presence of labeled peroxidase on the sections was visualized by incubation with the chromogenic substrate mix containing diaminobenzidine and H₂O₂. After careful rinsing, the sections were counterstained with Hematoxylin of Mayer and mounted with Entellan Neu (Merck, Amsterdam, The Nederlands). As control to exclude antigen-independent staining, the primary antibodies were either omitted or replaced by non-immune antisera. In all cases, these controls were negative.

The primary antibodies raised against the Na³⁰/K⁺-ATPase α1, α2 and α3 subunits were obtained from Upstate (Bio-Connect BV; Huissen; The Nederlands; α1 and α2) and from Sigma (Bornem, Belgium; α3).

After immunohistochemistry, the levels of the Na⁺/K⁺-ATPase α1, α2 and α3 subunit expression were quantitatively determined by using a computer-assisted KS 400 imaging system (Carl Zeiss vision, Hallbergmoos, Germany), as detailed previously (Saussez et al. 2006. Ann Surg Oncol 13: 999-1009). For each case we scanned 20 fields corresponding to surfaces ranging between 60,000 and 120,000 μm². Two independent persons analyzed 10 fields each for each case. The computer-assisted morphometric analysis of the parameters of immunohistochemical expression of each marker was quantitatively concerned the following two variables: 1) the Labeling Index (LI) which refers to the percentage of cells positively stained for a given marker and 2) the Mean Optical Density (MOD), which corresponds to the staining intensity of positive cells (Saussez et al. 2006, ibid).

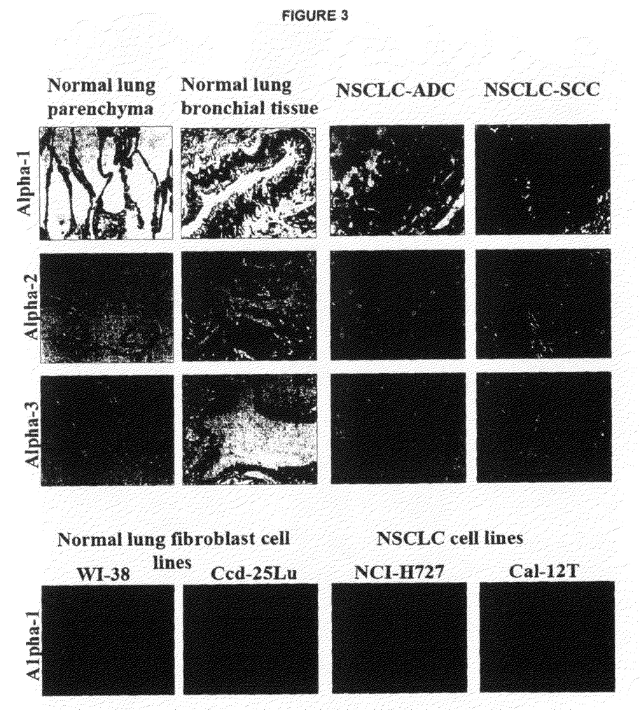

FIG. 3 morphologically illustrates the typical patterns of expression of the Na⁺/K⁺-ATPase α1, α2 and α3 subunits in normal lung parenchyma bronchial tissues vis-à-vis NSCLC-ADCs and NSCLC-SCCs. The increased expression of α1 in NSCLC-ADCs and its (α1)) strikingly high expression of in NSCLC-SCCs are immediately apparent.

The data from the present study strongly suggest an up-regulation of the Na⁺,K⁺-ATPase α1 subunit in a large proportion of NSCLCs as compared to normal lung tissues. Therefore, this Na⁺,K⁺-ATPase α1 subunit could be therapeutically targeted especially in those patients whose NSCLC overexpress it. In the sharp contrast, the Na⁺,K⁺-ATPase α2 subunit seems to be more expressed in normal lung tissues. The Na⁺/K⁺-ATPase α3 subunit seems expressed at rather low levels both in NSCLCs and in normal lung tissues in this particular experiment.

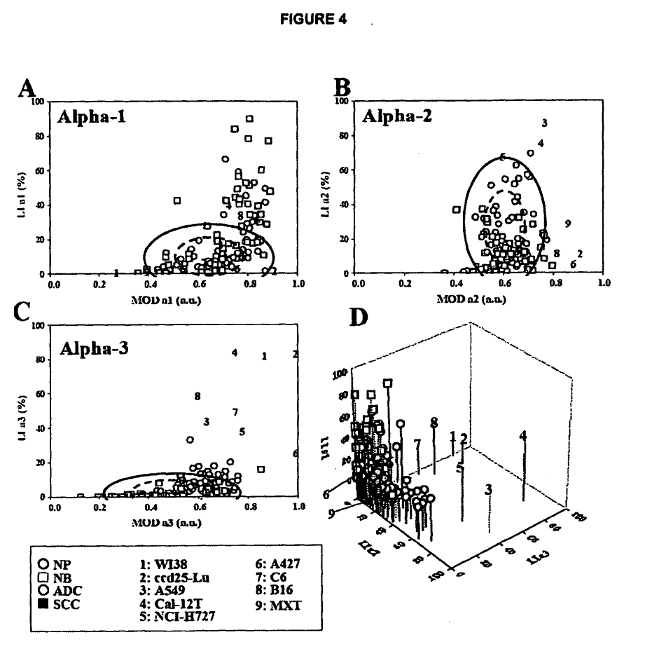

FIG. 4 illustrates the quantitative determination (performed by means of computer-assisted microscopy) of the immunohistochemical expression of the Na⁺,K⁺-ATPase α1, α2 and α3 subunits in the parenchyma (the open dots) and in the bronchial tissues (the open squares) of 25 normal lung tissues, in 30 NSCLC-ADCs (the filled dots) and in 29 NSCLC-SCCs (the filled squares). Numbers 1 and 2 represent two human normal lung fibroblast cell lines (WI-38 and ccd25-Lu), while numbers 3-6 represent human NSCLC cell lines (A549, Cal-12T, NCI-H727, A427). Numbers 7-9 represent three rodent tumor cell line, i.e. the rat C6 glioma (number 7), the mouse B16 melanoma (number 8) and the mouse MXT mammary carcinoma (number 9) models. Twenty quantitative measurements have been performed for each case and the mean LI (the percentages of cells expressing the marker) and the mean MOD (the concentration of the marker per cell (expressed in terms of optical densities)) values have been calculated from these 20 values, which therefore enabled each case to be located in a two-dimensional plane with its mean LI value on the Y axis and its MOD value on the X axis. The hatched elliptical lines represent the area including the mean +1×Sdev value calculated on all the 50 normal cases (the 25 parenchymal and the 25 bronchial tissues), while the full elliptical line represent the area including the mean +2×Sdev calculated on these 50 normal tissue samples.

FIG. 4A shows that 45/50 (90%) normal cases were included in the area delineated by the ellipse (the mean +2×Sdev) with respect to the immunohistochemical expression of the Na⁺,K⁺-ATPase al, while only 30/59 (50.85%) NSCLCs were included in this area. These data means that (and how) a threshold value can be defined in order to identify those patients whose NSCLC displays an immunohistochemical expression of the Na⁺/K⁺-ATPase α1 that is significantly higher than in normal lung tissues.

### Example 2

### siRNA Inhibition of Alpha-1 NKA Subunit Expression

Several anti-α1 subunit-siRNA-targeting nucleotides were designed and subsequently synthesised by Eurogentec (Seraing, Belgium) and evaluated for their ability to inhibit the Na⁺,K⁺-ATPase α1 subunit expression in human A549 NSCLC cells. The best results were obtained with the anti-α1 subunit siRNA with sense: 5′-GGGCAGUGUUUCAGGCUAA-3′ and anti-sense 5′-UUAGCCUGAAACACUGCCC-3′. A corresponding scrambled siRNA was used as a control (sense: 5′-UCUACGAGGCACGAGACUU-3′ and anti-sense: 5′-AACUCUCGUGCCUCGUAGA-3′. The anti-sense and sense strands of the siRNA were annealed by the manufacturer in 50 mM Tris, pH 7.5-8.0, 100 mM NaCl in DEPC-treated water. The final concentration of siRNA duplex was 100 μM. The anti-sense and sense strands of the scrambled control were annealed in the same way.

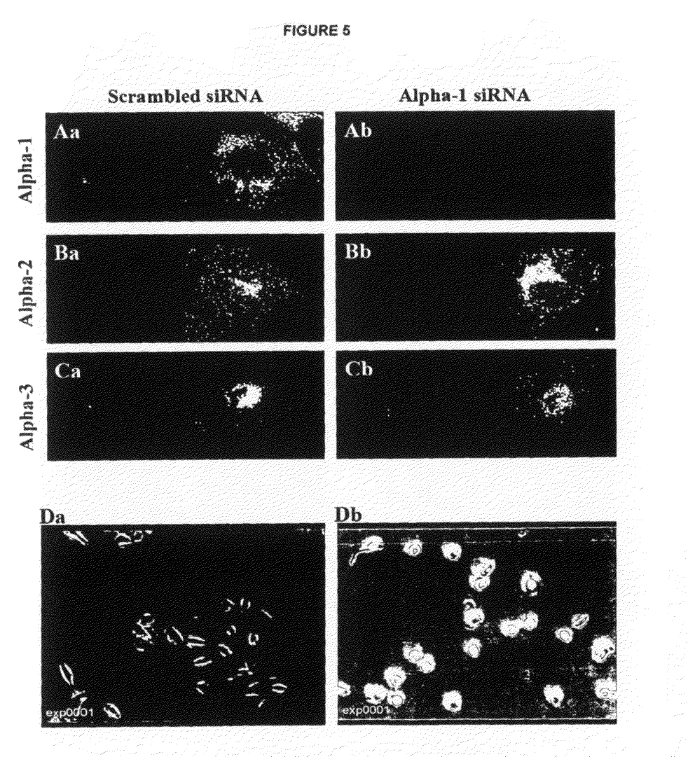

We made use of a siRNA targeting the Na⁺,K⁺-ATPase α1 subunit, whose efficiency is demonstrated in FIG. 5Ab. Depleting levels of expression of the Na⁺,K⁺-ATPase α1 subunit by means of this siRNA (“α1 siRNA”) did not modify the levels of expression of the Na⁺,K⁺-ATPase α2 (FIG. 5Bb) and α3 (FIG. 5Cb) subunits. No expression depletion was observed using the scrambled siRNA (FIGS. 5Aa, Ba, Ca). We observed by means of computer assisted video microscopy that a decrease by 80% of the Na⁺,K⁺-ATPase α1 subunit for 6 days using the present siRNA markedly impaired both A549 NSCLC cell proliferation and migration (FIG. 5Db), a feature not observed with the anti-al scrambled siRNA (FIG. 5Da).

### Example 3

### Sodium Pump Inhibition Assays in Insect Cells

Na⁺,K⁺-ATPase activity was assayed on homogenates of Sf-9 cells expressing the α1β1, α2β1 and α3β1 isozymes. The initial rate of release of ³²Pi from γ[³²P]-ATP was measured. The ATPase activity of 30-40 μg total protein samples was measured in a final volume of 0.25 ml in a medium containing 120 mM NaCl, 30 mM KCl, 3 mM MgCl2, 0.2 mM EGTA, 30 mM Tris-HCl (pH 7.4)±particular concentrations of a tested agent, e.g., herein compound 2.

Samples were preincubated with the inhibitor for 10 min. The assay was started by the addition of ATP with 0.2 μCi γ[32P]-ATP (2 mM final concentration). Following 30 min incubation at 37° C. the tubes were placed on ice, and the reaction was terminated by the addition of 25 μL of 55% trichloroacetic acid. Released 32Pi-Pi was converted into phosphomolybdate and extracted with isobutanol. The radioactivity in 150 μl of the organic phase was measured by liquid scintillation counting. The activity at 0.0001 M compound 2 was comparable to that obtained with 0.001 M ouabain. The specific activity was therefore determined as the difference in ATP hydrolysis in the presence and absence of 0.0001 M compound 2.

### Example 4

### Intracellular ATP ([ATP]i) Determination

Cellular ATP levels were measured by the bioluminescence assay according to the instructions accompanying an ATP determination kit (Molecular Probes, Invitrogen,

Merelbeke, Belgium). In brief, cells cultured in the presence or the absence of a tested agent, e.g., a presumed Na⁺,K⁺-ATPase inhibitor, e.g., herein compound 2 (10 nM), for the indicated periods were lysed with passive lysis buffer (Promega, Leiden, The Netherlands) and the protein concentration determined by the BCA (Pierce, Perbio Sciences, Erembodegem, Belgium) protein assay. Cell lysate (1 μg) was mixed with 100 μl of luciferase reagent and the luminescence analyzed on a luminometer TD-20/20 (Promega, Leiden, The Netherlands). The ATP concentration of the samples was calculated from a calibration curve for known ATP concentrations established at the same time. The data are expressed as percentage of treatment-induced decrease in cellular ATP concentration with the untreated, control condition set at 100%.

### Example 5

### Compound 2 Displays Significantly Higher Binding Affinity for the Na+/K+-ATPase α1 Subunit than Other Reference Cardenolides

We were wondering whether compound 2 could display significant binding affinity for the Na⁺/K⁺-ATPase α1 subunit as compared to other reference cardenolides such as ouabain and digoxin. We choose ouabain as a first cardenolide of reference because it is the cardenolide whose biological effects and signalling through the sodium pump have been the best characterized to date. We choose digoxin as a second cardenolide of reference because it is used to treat approximately 1.7 million patients in the USA each year for heart failure and/or atrial fibrillation despite the development of newer pharmacological agents such as angiotensin-converting enzyme (ACE) inhibitors, angiotensin II receptor antagonists and β-blockers.

To determine the effect of compound 2 on the activity of the Na⁺/K⁺-ATPase and its various isozymes, α1β1, α2β1 and α3β1 sodium pump complexes were produced using the baculovirus expression system and dose-response curves to the compound were performed. The use of heterologous protein expression in insect cells (example 3) has been used because it offers the advantage of an eukaryotic expression system able to produce large amounts of active recombinant Na⁺/K⁺-ATPase in a background with very little or no endogenous Na⁺/K⁺-ATPase present (Blanco 2005. Front Biosci 10: 2397-2411). Compound 2 has an inhibitory effect on all α1β1, α2β1 and α3β1 Na,K-ATPases (Table 4A). The calculated inhibition constants (Ki) showed that compound 2 inhibited α1β1 with a potency that is approximately 100 times greater than that of ouabain (Table 4A).

TABLE 4A

| Molecule | α1β1 | α2β1 | α3β1 |
|---|---|---|---|
| Compound 2 | 160 ± 70 | 15 ± 8 | 3.2 ± 3.4 |
| Digoxin | 930 ± 410 | 190 ± 90 | 40 ± 15 |
| Ouabain** | 43000 ± 19000 | 170 ± 10 | 31 ± 3 |

**Blanco et al. 2005 (supra)

### Example 6

### Compound 2 Displays Significantly Higher Anti-Proliferative Activity in Cancer than in Normal Cells and Significantly Higher Anti-Tumour Effects than Digoxin

The data from the example 4 strongly suggest that the α1 subunit of the sodium pump is a target for compound 2 and that compound 2 displays higher binding affinity for this α1 subunit than ouabain and digoxin. In order to functionally check this hypothesis we first made use of four human NSCLC cell lines on which we tested in vitro the anti-tumour effects (at the level of the cell population global growth development over three days) of compound 2, ouabain, digitoxin and digoxin. We also made use of three murine cell lines, i.e., three cancer cell lines including the C6 rat glioma, the MXT mouse mammary adenocarcinomas and the mouse B16F10 melanoma models.

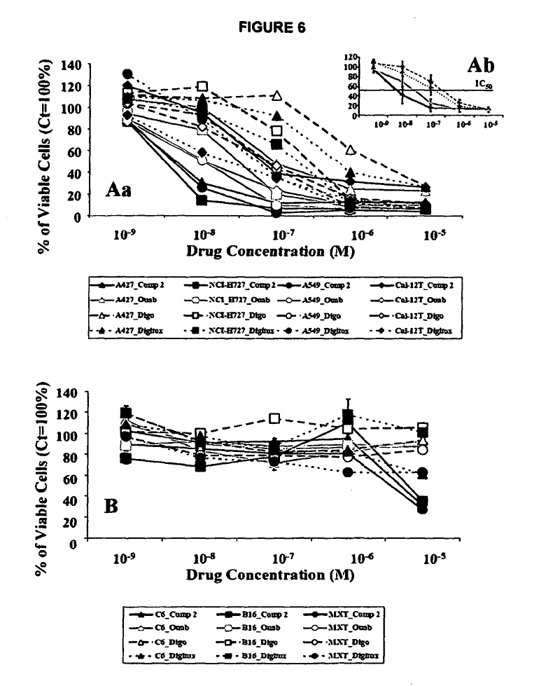

The data in FIG. 6Aa show that increased impairments in the global growth of the four human NSCLC cell lines occurred according to the sequence digoxin<digitoxin<ouabain<Compound 2, indicating that digoxin was the weakest and Compound 2 the strongest anti-tumor agent of these four cardenolides. FIG. 6Ab confirms this sequence when the mean growth curves (calculated on the four growth curves available for each product) are taken into account.

Then, we made use of three rodent cancer cell lines, i.e. the C6 rat glioma, the MXT mouse to mammary carcinoma and the mouse B16F10 melanoma models. The data in FIG. 6B show that apart from Compound 2 that displays actual anti-tumor effects, at high doses (10 μM), the remaining three cardenolides remained without any apparent anti-tumor effects. Depending on the species, the sodium pump α1 subunit exhibits different sensitivity to cardenolides. In rodents, α1 is between 100 and 1,000 times less sensitive to cardenolides than the α1 subunit from humans (because the α1 subunit in rodents displays two mutations that are not present in humans), whereas it is not the case with respect to the α2 and the α3 subunits.

Therefore, comparing the data in FIGS. 6A and 6B leads to the conclusions that i) it is the α1 subunit of the sodium that is mainly involved in the growth of tumor cells and ii) Compound 2 is more potent in inhibiting α1 subunit activity as compared to other well-known cardenolides such as ouabain, digitoxin or digoxin.

1. An antisense agent or an agent capable of causing RNA interference that can reduce the expression of the alpha-1 subunit of Na⁺,K⁺-ATPase (NKA), for use in the treatment of non-small-cell lung cancer (NSCLC), glioma, melanoma, lymphoma, gastrointestinal cancer, primitive neuroectodermal tumours (PNET), sarcoma, head & neck cancer, colon cancer, prostate cancer, pancreas cancer, ovary cancer, uterus cancer or breast cancer.

2. An antibody, fragment or derivative thereof, specific for the alpha-1 subunit of NKA, for use in the treatment of NSCLC, glioma, melanoma, lymphoma, gastrointestinal cancer, PNET, sarcoma, head & neck cancer, colon cancer, prostate cancer, pancreas cancer, ovary cancer, uterus cancer or breast cancer.

3. A method of treating NSCLC, glioma, melanoma, lymphoma, gastrointestinal cancer, PNET, sarcoma, head & neck cancer, colon cancer, prostate cancer, pancreas cancer, ovary cancer, uterus cancer or breast cancer comprising administering an antisense agent or an agent capable of causing RNA interference that can reduce the expression of the alpha-1 subunit of NKA according to claim 1 to an individual in need thereof.

4. A method of treating NSCLC, glioma, melanoma, lymphoma, gastrointestinal cancer, PNET, sarcoma, head & neck cancer, colon cancer, prostate cancer, pancreas cancer, ovary cancer, uterus cancer or breast cancer comprising administering an antibody, fragment or derivative thereof, specific for the alpha-1 subunit NKA according to claim 2 to an individual in need thereof.

5. A method comprising: (1) identifying or generating an antisense agent or an agent capable of causing RNA interference that can reduce the expression of the alpha-1 subunit of NKA or an antibody, fragment or derivative thereof, specific for the alpha-1 subunit of NKA; and (2) administering the agent of (1) to an individual in need thereof for the treatment of NSCLC, glioma, melanoma, lymphoma, gastrointestinal cancer, PNET, sarcoma, head & neck cancer, colon cancer, prostate cancer pancreas cancer, ovary cancer, uterus cancer or breast cancer.

6. The method according to claim 4, wherein said antibody, fragment or derivative thereof alters, preferably inhibits or activates, one or more aspects of the biological activity of NKA.

7. The antisense agent according to claim 1, wherein the antisense agent is an antisense oligonucleotide.

8. A pharmaceutical composition comprising a therapeutically effective amount of one or more of an antisense agent that can reduce the expression of NKA, an agent capable of causing RNA interference to reduce the expression of NKA, or an antibody, fragment or derivative thereof specific for the alpha-1 subunit of NKA, or a pharmaceutically acceptable salt thereof, and further comprising one or more of pharmaceutically acceptable buffers, carriers, excipients, stabilisers.

9. An assay to select, from a group of test agents, a candidate agent potentially, useful as a therapeutic in the treatment of NSCLC, glioma, melanoma, lymphoma, gastrointestinal cancer, PNET, sarcoma, head & neck cancer, colon cancer, prostate cancer, pancreas cancer, ovary cancer, uterus cancer or breast cancer, said assay comprising determining whether a tested agent (a) can reduce the expression of the alpha-1 subunit of Na⁺,K⁺-ATPase or (b) can bind to the alpha-1 subunit of Na⁺,K⁺-ATPase.

10. The assay of claim 9, further comprising use of the selected candidate agent for the preparation of a composition for administration to and monitoring the therapeutic effect thereof in a non-human animal model, preferably a non-human mammal model, of NSCLC, glioma, melanoma, lymphoma, gastrointestinal cancer, PNET, sarcoma, head & neck cancer, colon cancer, prostate cancer, pancreas cancer, ovary cancer, uterus cancer or breast cancer.

11. The method according to claim 3, wherein the agent is an antisense oligonucleotide.

12. The method according to claim 5, wherein the agent is an antisense oligonucleotide.
